(19) United States
(12) Patent Application Publication
(10) Pub. No.: US 2006/0251617 A1
(43) Pub. Date: Nov. 9, 2006

(54) Methods for treating lymphomas

(75) Inventors: Kimberly Denis-Mize, Concord, CA (US); Carla Heise, Benicia, CA (US); Daniel Menezes, Emeryville, CA (US); Susan E. Wilson, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/355,313

(22) Filed: Feb. 14, 2006

Related U.S. Application Data
(60) Provisional application No. 60/653,233, filed on Feb. 15, 2005.
(60) Provisional application No. 60/671,376, filed on Apr. 14, 2005.

(51) Int. Cl. A61K 38/20; A61K 39/395; A61K 31/704; A61K 31/57; A61K 31/66; A61K 31/4745

(52) U.S. Cl. 424/85.2; 424/155.1; 514/34; 514/109; 514/171; 514/283

(57) ABSTRACT

Methods for treating B-cell lymphomas, such as non-Hodgkin's lymphoma (NHL) are disclosed. The methods use a combination therapy of a chemotherapeutic agent, an IL-2 and, optionally, an anti-CD20 antibody.

### CROSS-REFERENCE TO RELATED APPLICATION

This application claims benefit under 35 U.S.C. § 119(e) of provisional application 60/653,233, filed Feb. 15, 2005, and provisional application 60/671,376, filed Apr. 14, 2005, which applications are hereby incorporated by reference in their entireties.

### TECHNICAL FIELD

The present invention pertains generally to methods for treating lymphomas. In particular, the invention relates to methods of treating B-cell lymphomas using a combination therapy of a chemotherapeutic agent, IL-2 and, optionally, an anti-CD20 antibody.

### BACKGROUND

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al., N. Engl. J. Med. (1987) 316:889-897; Rosenberg, Ann. Surg. (1988) 208:121-135; Topalian et al., J. Clin. Oncol. (1988) 6:839-853; Rosenberg et al., N. Engl. J. Med. (1988) 319:1676-1680; and Weber et al., J. Clin. Oncol. (1992) 10:33-40). The anti-tumor activity of IL-2 has best been described in patients with metastatic melanoma and renal cell carcinoma using Proleukin®, a commercially available IL-2 formulation from Chiron Corporation, Emeryville, Calif. Other diseases, including lymphoma, also appear to respond to treatment with IL-2 (Gisselbrecht et al., Blood (1994) 83:2020-2022).

Monoclonal antibodies have increasingly become a method of choice for the treatment of solid tumors such as breast cancer, as well as for treatment of lymphomas of the B-cell type, which express the CD20 cell surface antigen. In vitro work has demonstrated that monoclonal antibodies directed to CD20 result in cell death by apoptosis (Shan et al., Blood (1998) 91:1644-1652). Other studies report that B-cell death is primarily mediated by antibody-dependent cytotoxicity (ADCC).

Because of the possible immunological basis of the anti-tumor activity of anti-CD20 antibodies through ADCC mediated via NK cells, monocytes, macrophages and neutraphils, combinations with other cytokines that enhance NK cell function have been examined. Cytokines such as IL-12, IL-15, IL-21, TNF-alpha, TNF-beta, gamma-IFN, and IL-2 have been tested for potentiation of ADCC, a distinct NK function. All appear to be active in enhancing ADCC, although each agent is associated with its own specific toxicities. See, e.g., Rosenberg et al., Science (1986) 233:1318-1321; Gollob et al., J Clin Invest. (1998) 102:561-575. Ongoing studies of combination therapy with IL-2 and the monoclonal antibody rituximab (Rituxan®; IDEC-C2B8; IDEC Pharmaceuticals Corp., San Diego, Calif.) have shown improved clinical response in non-Hodgkin's B-cell lymphoma patients with these two therapeutic agents (U.S. Patent Publication 20030185796; Gluck et al., Clin. Cancer Res. (2004) 10:2253-2264).

Rituximab is a chimeric anti-CD20 monoclonal antibody containing human IgG1 and kappa constant regions with murine variable regions isolated from a murine anti-CD20 monoclonal antibody, IDEC-2B8 (Reff et al., Blood (1994) 83:435-445). Rituximab has been shown to be an effective treatment for low-intermediate and high-grade non-Hodgkin's lymphoma (NHL) (see, for example, Maloney et al., Blood (1994) 84:2457-2466; McLaughlin et al., J. Clin. Oncol. (1998) 16:2825-2833; Maloney et al., Blood (1997) 90:2188-2195; Hainsworth et. al., Blood (2000) 95:3052-3056; Colombat et al., Blood (2001) 97:101-106; Coiffier et al., Blood (1998) 92:1927-1932); Foran et al., J. Clin. Oncol. (2000) 18:317-324; Anderson et al., Biochem. Soc. Trans. (1997) 25:705-708; Vose et al., Ann. Oncol. (1999) 10:58a). However, 30% to 50% of patients with low-grade NHL exhibit no clinical response to this monoclonal antibody (Hainsworth et. al., Blood (2000) 95:3052-3056; Colombat et al., Blood (2001) 97:101-106). Though the exact mechanism of action is not known, evidence indicates that the anti-lymphoma effects of rituximab are in part due to complement mediated cytotoxicity (CMC), antibody-dependent cell mediated cytotoxicity (ADCC), inhibition of cell proliferation, and finally direct induction of apoptosis.

Non-Hodgkin's lymphoma comprises a group of lymphoid malignancies with an increasing incidence rate in both the United States and Europe. Low-grade and follicular lymphomas typically account for 40% of all NHL. Intermediate- and high-grade NHL patients respond well to chemotherapy, while low-grade and follicular lymphomas are difficult to treat, and patients can be refractory to current treatments or relapse post-treatment. Although most patients respond to initial chemotherapy, the course of disease results in progressively shorter remissions, highlighting the need for novel therapeutic strategies in NHL (Coiffier et al. (2004) Ann Hematol. 83 Suppl. 1:S73-4; Winter et al. (2004) Hematology (Am. Soc. Hematol. Educ. Program):203-220; Bendandi et al. (2004) Ann. Oncol. 15:703-11).

Thus, there remains a need for additional innovative strategies to improve the durability of clinical responses and overall tumor efficacy in B-cell lymphomas such as NHL.

### SUMMARY OF THE INVENTION

The present invention provides safe and efficacious methods for treating B-cell lymphomas, and in particular, NHLs. The methods utilize a combination of therapies, including the use of one or more chemotherapeutic agents, an IL-2 and, optionally, an anti-CD20 antibody. As shown in the examples herein, these therapeutic regimens significantly inhibit tumor growth and are superior to the use of individual CHOP constituents, rituximab, and CHOP and rituximab without IL-2.

In one aspect, the invention provides a method for treating a B-cell lymphoma comprising administering to a subject in need thereof a therapeutically effective amount of (a) one or more chemotherapeutic agents; (b) an IL-2; and, optionally, (c) an anti-CD20 antibody or antigen-binding fragment thereof.

In certain embodiments, the chemotherapeutic agent is selected from the group consisting of (a) cyclophosphamide, (b) doxorubicin, (c) vincristine, (d) prednisone and (e) combinations of cyclophosphamide, doxorubicin, vincristine and prednisone. In one embodiment, the chemotherapeutic agent comprises cyclophosphamide, doxorubicin, vincristine and prednisone (CHOP).

In certain embodiments, the antibody is an immunoglobulin G1 (IgG1) monoclonal antibody. In one embodiment, the antibody is rituximab.

In certain embodiments, the IL-2 is recombinantly produced IL-2 comprising an amino acid sequence having at least about 70%, preferably at least about 80%, more preferably at least about 90%, and most preferably at least about 95% sequence identity to the amino acid sequence of human IL-2. In one embodiment, the IL-2 is des-alanyl-1, serine-125 human interleukin-2 (aldesleukin).

In certain embodiments, multiple therapeutically effective doses of the IL-2 and the anti-CD20 antibody are administered to the subject. In certain embodiments, multiple therapeutically effective doses of the chemotherapeutic agent and the anti-CD20 antibody are administered to the subject. In certain embodiments, multiple therapeutically effective doses of the IL-2 and the anti-CD20 antibody are administered after administration of the chemotherapeutic agent and the anti-CD20 antibody. In certain embodiments, multiple therapeutically effective doses of the chemotherapeutic agent and the IL-2 are administered to the subject. In certain embodiments, multiple therapeutically effective doses of the IL-2 are administered after administration of the chemotherapeutic agent. In certain embodiments, multiple therapeutically effective doses of the IL-2 are administered to the subject for a time period sufficient to effect immune reconstitution. In certain embodiments, multiple therapeutically effective doses of the chemotherapeutic agent, the anti-CD20 antibody, and the IL-2 are administered to the subject.

In certain embodiments, IL-2 is administered according to a twice-a-week or three-times-a-week dosing regimen. In certain embodiments, the anti-CD20 antibody is administered according to a once-a-week dosing regimen. The IL-2 can be administered subcutaneously, the anti-CD20 antibody can be administered intraperitoneally or intravenously, and the chemotherapeutic agent can be administered intravenously or orally.

In certain embodiments, the invention provides a method for treating low grade/follicular non-Hodgkin's lymphoma (NHL). In one embodiment, the method comprises administering to a subject in need thereof therapeutically effective amounts of (a) CHOP; (b) des-alanyl-1, serine-125 human interleukin-2 (aldesleukin); and, optionally, (c) rituximab. Multiple therapeutically effective doses of CHOP, aldesleukin and/or rituximab can be administered to the subject. In certain embodiments, the aldesleukin is administered according to a twice-a-week or three-times-a-week dosing regimen. In one embodiment, the rituximab is administered according to a once-a-week dosing regimen. The aldesleukin can be administered subcutaneously, the rituximab can be administered intraperitoneally or intravenously, and the CHOP can be administered intravenously or orally.

These and other embodiments of the subject invention will readily occur to those of skill in the art in view of the disclosure herein.

### BRIEF DESCRIPTION OF THE FIGURES

FIGS. 1A-1D show mean tumor volume (mm³) in the human B-cell lymphoma (Daudi) model in BALB/c nude mice after the indicated treatment regimens.

FIGS. 2A-2C show time to tumor progression (conditional survival) of various treatment regimens in the human B-cell lymphoma (Daudi) model in BALB/c nude mice.

FIG. 3 shows the effects of IL-2+rituximab vs. rituximab or IL-2 or CHOP treatment on mean tumor volume (mm³) in the human B-cell lymphoma (Daudi) model in BALB/c nude mice.

FIG. 4 shows the effects of IL-2+rituximab vs. rituximab or IL-2 or CHOP treatment on % conditional survival in the human B-cell lymphoma (Daudi) model in BALB/c nude mice.

FIG. 5 shows the effects of IL-2+rituximab vs. CHOP+rituximab treatment on mean tumor volume (mm³) in the human B-cell lymphoma (Daudi) model in BALB/c nude mice.

FIG. 6 shows the effects of IL-2+rituximab vs. CHOP+rituximab treatment on % conditional survival in the human B-cell lymphoma (Daudi) model in BALB/c nude mice.

FIG. 7 shows the effects of CHOP+rituximab vs. rituximab or CHOP treatment on mean tumor volume (mm³) in the human B-cell lymphoma (Daudi) model in BALB/c nude mice.

FIG. 8 shows the effects of CHOP+rituximab vs. rituximab or CHOP treatment on % conditional survival in the human B-cell lymphoma (Daudi) model in BALB/c nude mice.

FIG. 9 shows the effects of CHOP+rituximab (day 8)+IL-2 (day 15) vs. CHOP+IL-2+rituximab (day 8) or CHOP+rituximab or IL-2+rituximab treatment on mean tumor volume (mm³) in the human B-cell lymphoma (Daudi) model in BALB/c nude mice.

FIG. 10 shows the effects of CHOP/rituximab+IL-2/rituximab vs. CHOP/IL-2/rituximab or CHOP/rituximab or IL-2/rituximab treatment on % conditional survival in the human B-cell lymphoma (Daudi) model in BALB/c nude mice.

FIG. 11 shows the effect of pretreatment with IL-2 prior to combination therapy of various treatment regimens on mean tumor volume (mm³) in the human B-cell lymphoma (Daudi) model in BALB/c nude mice.

FIG. 12 shows the effect of pretreatment with IL-2 prior to combination therapy of various treatment regimens on % conditional survival in the human B-cell lymphoma (Daudi) model in BALB/c nude mice.

FIG. 13 shows the effect of various treatment regimens on mean body weights in the human B-cell lymphoma (Daudi) model in BALB/c nude mice.

FIG. 14 shows the effect of combination treatment with IL-2 (3× weekly) and cyclophosphamide (C) (day 1) in the human B-cell lymphoma (Daudi) model in BALB/c nude mice.

FIG. 15 shows the effect of combination treatment with IL-2 (3× weekly) and doxorubicin (H) (day 1) in the human B-cell lymphoma (Daudi) model in BALB/c nude mice.

FIG. 16 shows the effect of combination treatment with IL-2 (3× weekly) and vincristine (O) (day 1) in the human B-cell lymphoma (Daudi) model in BALB/c nude mice.

FIG. 17 shows the effect of combination treatment with IL-2 (3× weekly) and prednisone (P) (qd×5) in the human B-cell lymphoma (Daudi) model in BALB/c nude mice.

FIG. 18 shows the effect of combination treatment with IL-2 (3× weekly) and CHOP in the human B-cell lymphoma (Daudi) model in BALB/c nude mice.

FIG. 19 shows the effect of CHOP therapy on monocyte and lymphocyte populations in the human B-cell lymphoma (Daudi) model in BALB/c nude mice (tumors approximately 300 mm³). CHOP was administered on day 1 and cell counts taken on day 4.

FIG. 20 shows the percentage of positive splenocytes from the human B-cell lymphoma (Daudi) model in BALB/c nude mice at day 15 after the indicated treatment regimens.

FIG. 21 shows the number of NK cells from whole blood (absolute counts) in the human B-cell lymphoma (Daudi) model in BALB/c nude mice at day 15 after the indicated treatment regimens.

FIG. 22 shows the number of activated monocytes from whole blood (absolute counts) in the human B-cell lymphoma (Daudi) model in BALB/c nude mice at day 15 after the indicated treatment regimens.

FIG. 23 shows CHOP+Rituximab+IL-2 therapy induces increased immune effector cells trafficking into tumors. Female BALB/c nude mice (6-8 weeks of age) bearing s.c. Daudi tumors (300 mm³) were treated on day 1 with either vehicle (5% dextrose) or CHOP administered alone or combined with rituximab (10 mg/kg on days 1, 8, 15). Treatment groups also included, CHOP-R followed by either vehicle (5% dextrose) or IL-2 (1 mg/kg on days 8, 10, 12, 15); or combined IL-2/R. On day 15, post treatment, tumors were collected and evaluated for histology and immunohistochemistry. Cellular trafficking of NK and monocytes into tumors: Panels: H&E stained (a-d); immunostained for perforin (e-h); F4/80 (i-1). All magnifications 400×, representative data, n=3-4 mice per group.

FIG. 24 shows CHOP+rituximab+IL-2 therapy induces increased apoptosis and potent anti-proliferative tumor responses in vivo. Female BALB/c nude mice (6-8 weeks of age) bearing s.c. Daudi tumors (300 mm³) were treated on day 1 with either vehicle (5% dextrose) or CHOP administered alone or combined with rituximab (10 mg/kg on days 1, 8, 15). Treatment groups also included CHOP+rituximab followed by either vehicle (5% dextrose) or IL-2 (1 mg/kg on days 8, 10, 12, 15); or combined IL-2 and rituximab. On day 15, post treatment, tumors were collected and evaluated for immunohistochemistry. Tumor cell apoptosis and proliferation were detected using cleaved caspase-3 (a-d) and Ki-67 (e-h), respectively. All magnifications 400×, representative data n=3-4 mice per group.

FIG. 25 depicts a schematic of administration schedules for all therapeutics.

FIGS. 26A and 26B shows that combination therapy with CHOP and IL-2 and rituximab is synergistic in the human Daudi lymphoma xenograft model. FIG. 26A shows tumor growth curves for groups treated with vehicle (♦), CHOP (“▪”), CHOP+IL-2 (-□-); CHOP+Rituximab (-Δ-); CHOP+IL-2+Rituximab (-•-). CHOP+Rituximab+IL-2 vs. CHOP+Rituximab; p<0.05. FIG. 26B shows Kaplan-Meier curves of conditional survival. Conditional survival was calculated as the time for each tumor to reach 1000 mm³ tumor volume. CHOP+Rituximab+IL-2 vs. CHOP+Rituximab; p=0.0002. Treatment groups: Vehicle (-♦-); Rituximab (-Δ-); CHOP (“▪”); CHOP+IL-2 (-□-); CHOP+Rituximab (-▴-); CHOP+Rituximab+IL-2 (-•-).

### DETAILED DESCRIPTION OF THE INVENTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Tumor Models in Cancer Research, (B. Teicher ed., Humana Press); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a chemotherapeutic agent” includes a mixture of two or more such agents, and the like.

The term “IL-2” as used herein is a protein derived from a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. Both full-length IL-2 proteins and biologically active fragments thereof are encompassed by the definition. The term also includes postexpression modifications of the IL-2, for example, glycosylation, acetylation, phosphorylation and the like. Furthermore, for purposes of the present invention, the term “IL-2” refers to a protein which includes modifications, such as deletions, additions and substitutions (generally conservative in nature), to the native sequence, so long as the protein maintains biological activity, i.e., anti-tumor activity when used in combination with a chemotherapeutic agent and, optionally, an anti-CD20 antibody. These modifications may be deliberate, as through site-directed mutagenesis, or may be accidental, such as through mutations of hosts which produce the proteins or errors due to PCR amplification.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule, that retain desired activity. For example, a variant, analog or mutein of IL-2 retains biological activity, such as anti-tumor activity, when used in combination with a chemotherapeutic agent and, optionally, an anti-CD20 antibody. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” refers to peptides having one or more peptide mimics (“peptoids”), such as those described in International Publication No. WO 91/04282. Preferably, the analog or mutein has at least the same biological activity as the native molecule. Methods for making polypeptide analogs and muteins are known in the art and are described further below.

The term also encompasses purposeful mutations that are made to the reference molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the protein of interest may include up to about 5-10 conservative or non-conservative amino acid substitutions, or even up to about 15-25, 50 or 75 conservative or non-conservative amino acid substitutions, or any integer between 5-75, so long as the desired function of the molecule remains intact. One of skill in the art can readily determine regions of the molecule of interest that can tolerate change.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, so long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the native polypeptide. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains biological activity, such as anti-tumor activity, as defined herein.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

An “antibody” intends a molecule that specifically binds to an epitope of interest present in an antigen. By “specifically binds” is meant that the antibody recognizes and interacts with the epitope in a “lock and key” type of interaction to form a complex between the antigen and antibody, as opposed to non-specific binding that might occur between the antibody and, for instance, a test substrate with which the antibody is reacted. The term “antibody” as used herein includes antibodies obtained from both polyclonal and monoclonal preparations, as well as, the following: hybrid (chimeric) antibody molecules (see, for example, Winter et al., Nature (1991) 349:293-299; and U.S. Pat. No. 4,816,567); F(ab′)2 and F(ab) fragments; Fv molecules (non-covalent heterodimers, see, for example, Inbar et al., Proc Natl Acad Sci USA (1972) 69:2659-2662; and Ehrlich et al., Biochem (1980) 19:4091-4096); single-chain Fv molecules (sFv) (see, for example, Huston et al., Proc Natl Acad Sci USA (1988) 85:5879-5883); dimeric and trimeric antibody fragment constructs; minibodies (see, e.g., Pack et al., Biochem (1992) 31:1579-1584; Cumber et al., J Immunology (1992) 149B: 120-126); humanized antibody molecules (see, for example, Riechmann et al., Nature (1988) 332:323-327; Verhoeyan et al., Science (1988) 239:1534-1536; and U.K. Patent Publication No. GB 2,276,169, published 21 Sep. 1994); and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

As used herein, the term “anti-cancer antibody” encompasses antibodies that have been designed to target cancer cells, particularly cell-surface antigens residing on cells of a particular cancer of interest. Preferably the anti-cancer antibody is monoclonal in nature, and preferably is an IgG1 monoclonal antibody.

As used herein, the term “anti-CD20 antibody” encompasses any antibody that specifically recognizes the CD20 B-cell surface antigen, including polyclonal anti-CD20 antibodies, monoclonal anti-CD20 antibodies, human anti-CD20 antibodies, humanized anti-CD20 antibodies, chimeric anti-CD20 antibodies, xenogeneic anti-CD20 antibodies, and fragments of these anti-CD20 antibodies that specifically recognize the CD20 B-cell surface antigen.

By “CD20 surface antigen” is intended a 33-37 kD integral membrane phosphoprotein that is expressed during early pre-B cell development and persists through mature B-cells but which is lost at the plasma cell stage. Although CD20 is expressed on normal B cells, this surface antigen is usually expressed at very high levels on neoplastic B cells. More than 90% of B-cell lymphomas and chronic lymphocytic leukemias, and about 50% of pre-B-cell acute lymphoblastic leukemias express this surface antigen.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using accepted animal models, such as the Namalwa and Daudi xenograft models of human B-cell lymphoma. See, e.g., Hudson et al., Leukemia (1998) 12:2029-2033 for a description of these animal models.

By “non-Hodgkin's B-cell lymphoma” or “NHL” is intended any of the non-Hodgkin's based lymphomas related to abnormal, uncontrollable B-cell proliferation. For purposes of the present invention, such lymphomas are referred to according to the Working Formulation classification scheme (see “The Non-Hodgkin's Lymphoma Pathologic Classification Project,” Cancer 49(1982):2112-2135), that is those B-cell lymphomas categorized as low grade, intermediate grade, and high grade. Low-grade B-cell lymphomas include small lymphocytic, follicular small-cleaved cell, and follicular mixed small-cleaved cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type.

By “therapeutically effective dose or amount” of IL-2 or variant thereof, or of an anti-CD20 antibody, is intended an amount that, when administered in combination with the chemotherapeutic agent, the IL-2 and the anti-CD20 antibody as described herein, brings about a positive therapeutic response, such as anti-tumor activity.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a novel therapeutic methodology for safely and effectively treating B-cell lymphomas, such as non-Hodgkin's B-cell lymphoma (NHL). The methods utilize delivery of a chemotherapeutic agent, an IL-2 and, optionally, an anti-CD20 antibody. Using these methods, the inventors herein have found that the overall anti-tumor effect, as well as durability of the response, is enhanced. In particular, as detailed in the examples, using a scientifically acceptable animal model of B-cell lymphoma, the inventors herein have shown that combined treatments of IL-2 and the chemotherapeutic agent CHOP, IL-2 and the individual constituents of CHOP, IL-2, CHOP and rituximab, as well as the IL-2 and the individual constituents of CHOP and rituximab, significantly inhibited tumor growth as compared to control treatments. Moreover, the time to tumor progression (TTP) was delayed, as compared to the use of the single agents IL-2 and rituximab. Strikingly, when IL-2 was added to the CHOP/rituximab regimen, substantial tumor regression was induced and TTP was increased, and the results were superior to the use of CHOP with rituximab alone.

The methods of the invention are useful in the therapeutic treatment of B-cell lymphomas that are classified according to the Revised European and American Lymphoma Classification (REAL) system. Such B-cell lymphomas include, but are not limited to, lymphomas classified as precursor B-cell neoplasms, such as B-lymphoblastic leukemia/lymphoma; peripheral B-cell neoplasms, including B-cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B-cell lymphoma (including extranodal, nodal, and splenic types), hairy cell leukemia, plasmacytoma/myeloma, diffuse large cell B-cell lymphoma of the subtype primary mediastinal (thymic), Burkitt's lymphoma, and Burkitt's like high grade B-cell lymphoma; and unclassifiable low-grade or high-grade B-cell lymphomas.

While the methods of the invention are directed to treatment of an existing lymphoma or solid tumor, it is recognized that the methods may be useful in preventing further tumor outgrowths arising during therapy. The methods of the invention are particularly useful in the treatment of subjects having low-grade B-cell lymphomas, particularly those subjects having relapses following standard chemotherapy. Low-grade B-cell lymphomas are more indolent than the intermediate- and high-grade B-cell lymphomas and are characterized by a relapsing/remitting course. Thus, treatment of these lymphomas is improved using the methods of the invention, as relapse episodes may be reduced in number and severity.

In order to further an understanding of the invention, a more detailed discussion is provided below regarding chemotherapeutic agents, anti-CD20 antibodies, IL-2, as well as modes of delivery of these substances.

IL-2

As explained above, the methods of the present invention include administering IL-2 in combination with a chemotherapeutic agent and, optionally, an anti-CD20 antibody. The IL-2 for use in the methods of the invention may be native or obtained by recombinant techniques, and may be from any source, including mammalian sources such as, e.g., mouse, rat, rabbit, primate, pig, and human. IL-2 sequences from a number of species are well known in the art and include but are not limited to, the following: human IL-2 (Homo sapiens; precursor sequence, GenBank Accession No. AAH66254; mature sequence represented by residues 21-153 of GenBank Accession No. AAH66254); rhesus monkey IL-2 (Macaca mulatto; precursor sequence, GenBank Accession No. P51498; mature sequence represented by residues 21-154 of GenBank Accession No. P51498 sequence); olive baboon IL-2 (Papio anubis; precursor sequence, GenBank Accession No. Q865Y1; mature sequence represented by residues 21-154 of GenBank Accession No. Q865Y1 sequence); sooty mangabey IL-2 (Cercocebus torquatus atys; precursor sequence, GenBank Accession No. P46649; mature sequence represented by residues 21-154 of GenBank Accession No. P46649 sequence); crab-eating macaque IL-2 (Macaca fascicularis; precursor sequence, GenBank Accession No. Q29615; mature sequence represented by residues 21-154 of GenBank Accession No. Q29615 sequence); common gibbon IL-2 (Hylobates lar; precursor sequence, GenBank Accession No. ICGI2; mature sequence represented by residues 21-153 of GenBank Accession No. ICGI2 sequence); common squirrel monkey IL-2 (Saimiri sciureus; precursor sequence, GenBank Accession No. Q8MKH2; mature sequence represented by residues 21-154 of GenBank Accession No. Q8MKH2 sequence); cow IL-2 (Bos taurus; precursor sequence, GenBank Accession No. P05016; mature sequence represented by residues 21-155 of GenBank Accession No. P05016 sequence; see also the variant precursor sequence reported in GenBank Accession No. NP-851340; mature sequence represented by residues 24-158 of GenBank Accession No. NP-851340 sequence); water buffalo IL-2 (Bubalus bubalis; precursor sequence, GenBank Q95 KP3; mature sequence represented by residues 21-155 of GenBank Q95 KP3 sequence); horse IL-2 (Equus caballus; precursor sequence, GenBank Accession No. P37997; mature sequence represented by residues 21-149 of GenBank Accession No. P37997 sequence); goat IL-2 (Capra hircus; precursor sequence, GenBank Accession No. P36835; mature sequence represented by residues 21-155 of GenBank Accession No. P36835 sequence); sheep IL-2 (Ovis aries; precursor sequence, GenBank Accession No. P19114; mature sequence represented by residues 21-155 of GenBank Accession No. P19114 sequence); pig IL-2 (Sus scrofa; precursor sequence, GenBank Accession No. P26891; mature sequence represented by residues 21-154 of GenBank Accession No. P26891); red deer IL-2 (Cervus elaphus; precursor sequence, GenBank Accession No. P51747; mature sequence represented by residues 21-162 of GenBank Accession No. P51747 sequence); dog IL-2 (Canis familiaris; precursor sequence, GenBank Accession No. Q29416; mature sequence represented by residues 21-155 of GenBank Accession No. Q29416 sequence); cat IL-2 (Felis catus; precursor sequence, GenBank Accession No. Q07885; mature sequence represented by residues 21-154 of GenBank Accession No. Q07885 sequence); rabbit IL-2 (Oryctolagus cuniculus; precursor sequence, GenBank Accession No. O77620; mature sequence represented by residues 21-153 of GenBank Accession No. O77620 sequence); killer whale IL-2 (Orcinus orca; precursor sequence, GenBank Accession No. 097513; mature sequence represented by residues 21-152 of GenBank Accession No. 097513 sequence); northern elephant seal IL-2 (Mirounga angustirostris; precursor sequence, GenBank Accession No. O62641; mature sequence represented by residues 21-154 of GenBank Accession No. O62641 sequence); house mouse IL-2 (Mus musculus; precursor sequence, GenBank Accession No. NP_{—}032392; mature sequence represented by residues 21-169 of GenBank Accession No. NP_{—}032392 sequence); western wild mouse IL-2 (Mus spretus; precursor sequence, GenBank Accession No. Q08867; mature sequence represented by residues 21-166 of GenBank Accession No. Q08867 sequence); Norway rat IL-2 (Rattus norvegicus; precursor sequence, GenBank Accession No. P17108; mature sequence represented by residues 21-155 of GenBank Accession No. P17108); Mongolian gerbil IL-2 (Meriones unguiculatus; precursor sequence, GenBank Accession No. Q08081; mature sequence represented by residues 21-155 of GenBank Accession No. Q08081); any of the variant IL-2 polypeptides disclosed in these foregoing GenBank Accession Numbers; each of which GenBank reports are herein incorporated by reference in their entirety. Though any source of IL-2 can be utilized to practice the invention, preferably the IL-2 is derived from a human source, particularly when the subject undergoing therapy is a human. In some embodiments, the IL-2 for use in the methods of the invention is recombinantly produced, for example, recombinant human IL-2 proteins, including, but not limited to, those obtained from microbial hosts.

The compositions useful in the methods of the invention may comprise biologically active variants of IL-2, including variants of IL-2 from any species. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

Suitable biologically active variants of native or naturally occurring IL-2 can be fragments, analogs, and derivatives of that polypeptide, as defined above.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor Laboratory Press, Plainview, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

Guidance as to regions of the IL-2 protein that can be altered either via residue substitutions, deletions, or insertions can be found in the art. See, for example, the structure/function relationships and/or binding studies discussed in Bazan (1992) Science 257:410-412; McKay (1992) Science 257:412; Theze et al. (1996) Immunol. Today 17:481-486; Buchli and Ciardelli (1993) Arch. Biochem. Biophys. 307:411-415; Collins et al. (1988) Proc. Natl. Acad. Sci. USA 85:7709-7713; Kuziel et al. (1993) J. Immunol. 150:5731; Eckenberg et al. (1997) Cytokine 9:488-498; the contents of which are herein incorporated by reference in their entirety.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Biologically active variants of IL-2 will generally have at least about 70%, preferably at least about 80%, more preferably at least about 90% to 95% or more, and most preferably at least about 98%, 99% or more amino acid sequence identity to the amino acid sequence of the reference IL-2 polypeptide molecule, such as native human IL-2, which serves as the basis for comparison. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2:482-489. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have the same number of amino acids, additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm). A biologically active variant of a native IL-2 polypeptide of interest may differ from the native polypeptide by as few as 1-15 amino acids, as few as 1-10, such as 6-10, as few as 5, as few as 4, 3, 2, or even 1 amino acid residue.

The precise chemical structure of a polypeptide having IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 or variants thereof for use in the methods of the present invention may be from any source, but preferably is recombinantly produced. By “recombinant IL-2” or “recombinant IL-2 variant” is intended interleukin-2 or variant thereof that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, and most preferably E. coli, as described herein. The host organism expresses the foreign gene to produce IL-2 under expression conditions. Synthetic recombinant IL-2 can also be made in eukaryotes, such as yeast or human cells. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of variant IL-2 proteins, see European Patent (EP) Publication No. EP 136,489 (which discloses one or more of the following alterations in the amino acid sequence of naturally occurring IL-2: Asn26 to Gln26; Trp121 to Phe121; Cys58 to Ser58 or Ala58, Cys105 to Ser105 or Ala105; Cys125 to Ser125 or Ala125; deletion of all residues following Arg 120; and the Met-1 forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL-2). See also U.S. Pat. No. 4,752,585 (which discloses the following variant IL-2 proteins: ala104 ser125 IL-2, ala104 IL-2, ala104 ala125 IL-2, val104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, des-ala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, des-ala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 IL-2, des-ala1 des-pro2 des-thr3 des-ser4 ala104 ser125 IL-2, des-ala1 des-pro2 des-thr3 des-ser4 ala104 IL-2, des-ala1 des-pro2 des-thr3 des-ser4 ala104 ala125 IL-2, des-ala1 des-pro2 des-thr3 des-ser4 val104 ser125 IL-2, des-ala1 des-pro2 des-thr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 IL-2, des-ala1 des-pro2 des-thr3 des-ser4 des-ser5 val104 ser125 IL-2, des-ala1 des-pro2 des-thr3 des-ser4 des-ser5 val 104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL-2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala1 des-pro2 des-thr3 des-ser4 des-ser5 des-ser6 val04 ala125 IL-2) and U.S. Pat. No. 4,931,543 (which discloses the IL-2 mutein des-alanyl-1, serine-125 human IL-2 used in the examples herein, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des-ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 des-thr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; des-ala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 variants bearing alanine instead of methionine as the N-terminal amino acid as found in the native molecule; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include those disclosed in WO 99/60128 (substitutions of the aspartate at position 20 with histidine or isoleucine, the asparagine at position 88 with arginine, glycine, or isoleucine, or the glutamine at position 126 with leucine or gulatamic acid), which reportedly have selective activity for high affinity IL-2 receptors expressed by cells expressing T cell receptors in preference to NK cells and reduced IL-2 toxicity; the muteins disclosed in U.S. Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine), which are claimed to reduce vascular leak syndrome; the IL-2 p1-30 peptide disclosed in International Publication No. WO 00/04048 (corresponding to the first 30 amino acids of IL-2, which contains the entire a-helix A of IL-2 and interacts with the b chain of the IL-2 receptor), which reportedly stimulates NK cells and induction of LAK cells; and a mutant form of the IL-2 p1-30 peptide also disclosed in WO 00/04048 (substitution of aspartic acid at position 20 with lysine), which reportedly is unable to induce vascular bleeds but remains capable of generating LAK cells. Additionally, IL-2 can be modified with polyethylene glycol to provide enhanced solubility and an altered pharmokinetic profile (see U.S. Pat. No. 4,766,106).

Additional examples of IL-2 muteins with predicted reduced toxicity are disclosed in U.S. Provisional Application Ser. No. 60/550,868, filed Mar. 5, 2004, herein incorporated by reference in its entirety. These muteins comprise the amino acid sequence of mature human IL-2 with a serine substituted for cysteine at position 125 of the mature human IL-2 sequence and at least one additional amino acid substitution within the mature human IL-2 sequence such that the mutein has the following functional characteristics: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, I24L, K32A, K32W, N33E, P34E, P34R, P34S, P34T, P34V, K35D, K351, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I, E61K, E61M, E61R, E62T, E62Y, K64D, K64E, K64G, K64L, K64Q, K64R, P65D, P65E, P65F, P65G, P65H, P65I, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L941, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q126I, and Q126V; where the amino acid residue position is relative to numbering of the mature human IL-2 amino acid sequence. In other embodiments, these muteins comprise the amino acid sequence of mature human IL-2 with an alanine substituted for cysteine at position 125 of the mature human IL-2 sequence and at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, I24L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q126I, and Q126V; where the amino acid residue position is relative to numbering of the mature human IL-2 amino acid sequence. In alternative embodiments, these muteins comprise the amino acid sequence of mature human IL-2 with at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, I24L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I, E61K, E61M, E61R, E62T, E62Y, K64D, K64E, K64G, K64L, K64Q, K64R, P65D, P65E, P65F, P65G, P65H, P65I, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q126I, and Q126V; where the amino acid residue position is relative to numbering of the mature human IL-2 amino acid sequence. Additional muteins disclosed in U.S. Provisional Application Ser. No. 60/550,868 include the foregoing identified muteins, with the exception of having the initial alanine residue at position 1 of the mature human IL-2 sequence deleted.

The term IL-2 as used herein is also intended to include IL-2 fusions or conjugates comprising IL-2 fused to a second protein or covalently conjugated to polyproline or a water-soluble polymer to reduce dosing frequencies or to improve IL-2 tolerability. For example, the IL-2 (or a variant thereof as defined herein) can be fused to human albumin or an albumin fragment using methods known in the art (see WO 01/79258). Alternatively, the IL-2 can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

Any pharmaceutical composition comprising IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997; herein incorporated by reference. Thus liquid, lyophilized, or spray-dried compositions comprising IL-2 or variants thereof that are known in the art may be prepared as an aqueous or nonaqueous solution or suspension for subsequent administration to a subject in accordance with the methods of the invention. Each of these compositions will comprise IL-2 or variants thereof as a therapeutically or prophylactically active component. By “therapeutically or prophylactically active component” is intended the IL-2 or variants thereof is specifically incorporated into the composition to bring about a desired therapeutic or prophylactic response with regard to treatment or prevention of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, the IL-2 containing pharmaceutical compositions useful in the methods of the invention are compositions comprising stabilized monomeric IL-2 or variants thereof, compositions comprising multimeric IL-2 or variants thereof, and compositions comprising stabilized lyophilized or spray-dried IL-2 or variants thereof.

Pharmaceutical compositions comprising stabilized monomeric IL-2 or variants thereof are disclosed in PCT application No. PCT/US00/27156, filed Oct. 3, 2000, the disclosure of which is herein incorporated by reference. By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 or variants thereof in these liquid compositions is formulated with an amount of an amino acid base sufficient to decrease aggregate formation of IL-2 or variants thereof during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2 or variants thereof, where the buffering agent is an acid substantially free of its salt form, an acid in its salt form, or a mixture of an acid and its salt form. Preferably the acid is selected from the group consisting of succinic acid, citric acid, phosphoric acid, and glutamic acid. Such compositions are referred to herein as stabilized monomeric IL-2 pharmaceutical compositions.

The amino acid base in these compositions serves to stabilize the IL-2 or variants thereof against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 or variants thereof may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Other examples of IL-2 formulations that comprise IL-2 in its nonaggregated monomeric state include those described in Whittington and Faulds (1993) Drugs 46(3):446-514. These formulations include the recombinant IL-2 product in which the recombinant IL-2 mutein Teceleukin (unglycosylated human IL-2 with a methionine residue added at the amino-terminal) is formulated with 0.25% human serum albumin in a lyophilized powder that is reconstituted in isotonic saline, and the recombinant IL-2 mutein Bioleukin (human IL-2 with a methionine residue added at the amino-terminal, and a substitution of the cysteine residue at position 125 of the human IL-2 sequence with alanine) formulated such that 0.1 to 1.0 mg/ml IL-2 mutein is combined with acid, wherein the formulation has a pH of 3.0 to 4.0, advantageously no buffer, and a conductivity of less than 1000 mmhos/cm (advantageously less than 500 mmhos/cm). See EP 373,679; Xhang et al. (1996) Pharmaceut. Res. 13(4):643-644; and Prestrelski et al. (1995) Pharmaceut. Res. 12(9):1250-1258.

Examples of pharmaceutical compositions comprising multimeric IL-2 or variants thereof are disclosed in commonly owned U.S. Pat. No. 4,604,377, the disclosure of which is herein incorporated by reference. By “multimeric” is intended the protein molecules are present in the pharmaceutical composition in a microaggregated form having an average molecular association of 10-50 molecules. These multimers are present as loosely bound, physically-associated IL-2 molecules. A lyophilized form of these compositions is available commercially under the tradename Proleukin® (Chiron Corporation, Emeryville, Calif.). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 or variants thereof retains its multimeric state. Such lyophilized or liquid compositions comprising multimeric IL-2 or variants thereof are encompassed by the methods of the present invention. Such compositions are referred to herein as multimeric IL-2 pharmaceutical compositions.

The methods of the present invention may also use stabilized lyophilized or spray-dried pharmaceutical compositions comprising IL-2 or variants thereof, which may be reconstituted into a liquid or other suitable form for administration in accordance with methods of the invention. Such pharmaceutical compositions are disclosed in copending application U.S. Ser. No. 09/724,810, filed Nov. 28, 2000 and International Application PCT/US00/35452, filed Dec. 27, 2000, herein incorporated by reference in their entireties. These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 or variants thereof against methionine oxidation. Use of these agents in this manner is described in copending U.S. Provisional Application Ser. No. 60/157,696, herein incorporated by reference. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising IL-2 or variant thereof as a therapeutically active component is encompassed by the methods of the invention.

Chemotherapeutic Agents

The combined therapeutic methods of the present invention further comprise administration of at least one chemotherapeutic agent or regimen. A “chemotherapeutic agent” is a chemical compound or combination of compounds useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembiehin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofuran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and doxetaxel (TAXOTEW, Rhône-Poulenc Rorer, Antony, France); gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above.

Other useful chemotherapeutic agents include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Particularly useful are the chemotherapeutic regimens known as CHOP (a combination of cyclophosphamide, doxorubicin, vincristine and prednisone) as well as the use of the constituents of CHOP either alone or in various combinations such as CO, CH, CP, COP, CHO, CHP, HO, HP, HOP, OP, etc.; CHOP and bleomycin (CHOP-BLEO); cyclophosphamide and fludarabine; cyclophosphamide, mitoxantrone, prednisone and vincristine; cyclophosphamide, dexamethasone, doxorubicin and vincristine (CAVD); CAV; cyclophosphamide, doxorubicin and prednisone; cyclophosphamide, mitoxantrone, prednisone and vincristine (CNOP); cyclophosphamide, methotrexate, leucovorin and cytarabine (COMLA); cyclophosphamide, dexamethasone, doxorubicin and prednisone; cylophosphamide, prednisone, procarbazine and vincristine (COPP); cylophosphamide, prednisone and vincristine (COP and CVP-1); cyclophosphamide and mitoxantrone; etoposide; mitoxantrone, ifosfamide and etoposide (MIV); cytarabine; methylprednisolone and cisplatin (ESHAP); methylprednisolone, cytarabine and cisplatin (ESAP); methotrexate, leucovorin, doxorubicin, cyclophosphamide, vincristine, bleomycin and prednisone (MACOP-B); methotrexate, bleomycin, doxorubicin, cyclophosphamide, vincristine, and dexamethasone (m-BACOD); prednisone, cyclophosphamide, etoposide, cytarabine, bleomycin, vincristine, methotrexate and leucovorin (PROMACE-CYTABOM); etoposide, cyclophosphamide, vincristine, prednisone and bleomycin (VACOP-B); fludarabine and mitoxantrone; cisplatine, cytarabine and etoposide; desamethasone, fludarabine and mitoxantrone; chlorambucil and prednisone; busulfan and fludarabine; ICE; DVP; ATRA; Idarubicin, hoelzer chemotherapy regime; La La chemotherapy regime; ABVD; CEOP; 2-CdA; FLAG and IDA (with or without subsequent G-CSF treatment); VAD; M and P; C-Weekly; ABCM; MOPP; cisplatin, cytarabine and dexamethasone (DHAP), as well as the additional known chemotherapeutic regimens. A preferred chemotherapeutic regimen for the treatment of non-Hodgkin's lymphoma patients is CHOP.

Anti-CD20 Antibodies

As explained above, an anti-CD20 antibody can be administered in combination with IL-2 therapy and a chemotherapeutic agent. Particularly useful are antibodies that mediate their cytotoxicity effects via IgG1/FcγR-mediated ADCC. Such antibodies include, but are not limited to, Rituxan® (which targets the CD20 antigen on neoplastic B cells, and is effective for treatment of B-cell lymphomas, including non-Hodgkin's B-cell lymphomas, and chronic lymphocytic leukemia (CLL)); and other anti-CD20 antibodies such as Hu-MAX-CD20, IMMU-106, TRU-015 including those that have been engineered for increased ADCC activity. Also useful is Zevalin a radioimmunotherapeutic that comprises a murine monoclonal antibody (ibritumomab) bound to a radioactive isotope (yttrium-90) by a strong linking agent (tiuxetan), used in combination with Rituxan®. The Zevalin therapeutic regimen comprises an infusion of Rituxan® preceding an injection of the Zevalin antibody linked to the indium-111 radioisotope, followed seven to nine days later by a second infusion of Rituxan® prior to an injection of Zevalin linked to the yttrium-90 radioisotope(dosed at 0.4 mCi/kg body weight). The BEXXAR radioimmunotherapeutic regimen using Tositumomab (anti-CD20) and Iodine I-131 Tositumomab can also be used in the subject methods.

As used herein, the term “anti-CD20 antibody” encompasses any antibody that specifically recognizes the CD20 B-cell surface antigen, including polyclonal anti-CD20 antibodies, monoclonal anti-CD20 antibodies, human anti-CD20 antibodies, humanized anti-CD20 antibodies, chimeric anti-CD20 antibodies, xenogeneic anti-CD20 antibodies, and fragments of these anti-CD20 antibodies that specifically recognize the CD20 B-cell surface antigen. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, i.e., the CD20 B-cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

Anti-CD20 antibodies of murine origin are suitable for use in the methods of the present invention. Examples of such murine anti-CD20 antibodies include, but are not limited to, the B1 antibody (described in U.S. Pat. No. 6,015,542); the 1F5 antibody (see Press et al. (1989) J. Clin. Oncol. 7:1027); NKI-B20 and BCA-B20 anti-CD20 antibodies (described in Hooijberg et al. (1995) Cancer Research 55:840-846); and IDEC-2B8 (available commercially from IDEC Pharmaceuticals Corp., San Diego, Calif.); the 2H7 antibody (described in Clark et al. (1985) Proc. Natl. Acad. Sci. USA 82:1766-1770; and others described in Clark et al. (1985) supra and Stashenko et al. (1980) J. Immunol. 125:1678-1685.

The term “anti-CD20 antibody” as used herein encompasses chimeric anti-CD20 antibodies. By “chimeric antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD20 cell surface antigen. The non-human source can be any vertebrate source that can be used to generate antibodies to a human CD20 cell surface antigen or material comprising a human CD20 cell surface antigen. Such non-human sources include, but are not limited to, rodents (e.g., rabbit, rat, mouse, etc.; see, for example, U.S. Pat. No. 4,816,567) and non-human primates (e.g., Old World Monkey, Ape, etc.; see, for example, U.S. Pat. Nos. 5,750,105 and 5,756,096). Most preferably, the non-human component (variable region) is derived from a murine source. As used herein, the phrase “immunologically active” when used in reference to chimeric anti-CD20 antibodies means a chimeric antibody that binds human C1q, mediates complement dependent lysis (“CDC”) of human B lymphoid cell lines, and lyses human target cells through antibody dependent cellular cytotoxicity (“ADCC”). Examples of chimeric anti-CD20 antibodies include, but are not limited to, IDEC-C2B8, available commercially under the name rituximab (Rituxan®; IDEC Pharmaceuticals Corp., San Diego, Calif.) and described in U.S. Pat. Nos. 5,736,137, 5,776,456, and 5,843,439, all of which patents are incorporated herein by reference in their entireties; the chimeric antibodies described in U.S. Pat. No. 5,750,105; those described in U.S. Pat. Nos. 5,500,362; 5,677,180; 5,721,108; and 5,843,685, all of which patents are incorporated herein by reference in their entireties.

Humanized anti-CD20 antibodies are also encompassed by the term anti-CD20 antibody as used herein. By “humanized” is intended forms of anti-CD20 antibodies that contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596.

Also encompassed by the term anti-CD20 antibodies are xenogeneic or modified anti-CD20 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598.

Fragments of the anti-CD20 antibodies are suitable for use in the methods of the invention so long as they retain the desired affinity of the full-length antibody. Thus, a fragment of an anti-CD20 antibody will retain the ability to bind to the CD20 B-cell surface antigen. Fragments of an antibody comprise a portion of a full-length antibody, generally the antigen binding or variable region thereof. Examples of antibody fragments include, but are not limited to, Fab, Fab′, F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Further, any of the previously described anti-CD20 antibodies may be conjugated prior to use in the methods of the present invention. Such conjugated antibodies are available in the art. Thus, the anti-CD20 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603. Alternatively, the anti-CD20 antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred. See also, for example, labeled forms of anti-CD20 antibodies described in U.S. Pat. No. 6,015,542.

The anti-CD20 antibodies are typically provided by standard techniques within a pharmaceutically acceptable buffer, for example, sterile saline, sterile buffered water, propylene glycol, combinations of the foregoing, etc. Methods for preparing parenterally administerable agents are described in Remington's Pharmaceutical Sciences (18^{th} ed.; Mack Pub. Co.: Eaton, Pa., 1990). See also, for example, International Publication No. WO 98/56418, which describes stabilized antibody pharmaceutical formulations suitable for use in the methods of the present invention. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5. Lyophilized formulations adapted for subcutaneous administration are described in International Publication No. WO97/04801. Such lyophilized formulations may be reconstituted with a suitable diluent to a high protein concentration and the reconstituted formulation may be administered subcutaneously to the mammal to be treated herein.

Administration

At least one therapeutically effective dose of the chemotherapeutic agent, IL-2 or variant thereof, and, optionally, an anti-CD20 antibody, will be administered. By “therapeutically effective dose or amount” of each of these agents is intended an amount that when administered in combination with the other agents, brings about a positive therapeutic response with respect to treatment of an individual for a B-cell lymphoma, particularly NHL. Of particular interest is an amount of these agents that provides an anti-tumor effect, as defined herein. By “positive therapeutic response” is intended the individual undergoing the combination treatment according to the invention exhibits an improvement in one or more symptoms of the B-cell lymphoma for which the individual is undergoing therapy.

Thus, for example, a “positive therapeutic response” would be an improvement in the disease in association with the combination therapy, and/or an improvement in one or more symptoms of the disease in association with the combination therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

In certain embodiments, multiple therapeutically effective doses of each of the chemotherapeutic agent, the IL-2 or variant thereof, and optionally, the anti-CD20 antibody will be administered using therapeutically active regimens. IL-2 may be administered according to a daily dosing regimen, or intermittently. By “intermittent” administration is intended the therapeutically effective dose can be administered, for example, every other day, every two days, every three days, and so forth. For example, in some embodiments, IL-2 will be administered twice-weekly or thrice-weekly for an extended period of time, such as for 1, 2, 3, 4, 5, 6, 7, 8 . . . 10 . . . 15 weeks, and so forth. The anti-CD20 antibody will be administered intermittently, for example, once weekly or twice weekly, with repeated cycles. In some embodiments, the anti-CD20 antibody will be administered once or twice-weekly for an extended period of time, such as for 1, 2, 3, or 4 weeks. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

The IL-2 or variant thereof can be administered prior to, concurrent with, or subsequent to the chemotherapeutic agent and/or the anti-CD20 antibody. For example, initial treatment with the chemotherapeutic agent, such as CHOP and the anti-CD20 antibody can be performed, followed by one or more treatments with IL-2 and the anti-CD20 antibody. If provided at the same time as the chemotherapeutic agent and/or the anti-CD20 antibody, the IL-2 or variant thereof can be provided in the same or in a different composition. Thus, the three agents, or two of the three agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising IL-2 or a variant thereof and the chemotherapeutic agent, such as CHOP, can be administered in at least one therapeutic dose. Similarly, at least one therapeutically effective dose of a pharmaceutical composition comprising at least one anti-CD20 antibody or antigen-binding fragment thereof can be administered according to a particular dosing regimen. Administration of separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

In other embodiments of the invention, the pharmaceutical compositions comprising the agents, such as IL-2 or variant thereof, is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the chemotherapeutic agent or agents, anti-CD20 antibody and the IL-2 or variant thereof may be administered using the same or different routes of administration in accordance with any medically acceptable method known in the art. Suitable routes of administration include parenteral administration, such as subcutaneous (s.c.), intraperitoneal (i.p.), intramuscular (i.m.), intravenous (i.v.), or infusion, oral (p.o.) and pulmonary, nasal, topical, transdermal, and suppositories. Where the composition is administered via pulmonary delivery, the therapeutically effective dose is adjusted such that the soluble level of the agent, such as the IL-2 or variant thereof in the bloodstream, is equivalent to that obtained with a therapeutically effective dose that is administered parenterally, for example s.c., i.p., i.m., or i.v. In some embodiments of the invention, the pharmaceutical composition comprising IL-2 or a variant thereof is administered by i.m. or s.c. injection, particularly by i.m. or s.c. injection locally to the region where the therapeutic agent or agents used in the cancer therapy protocol are administered. Similarly, the anti-CD20 antibody can be administered by i.v., i.m., i.p. or s.c. injection. In a particularly preferred embodiment, the chemotherapeutic agent is administered, for example, intravenously. When administered intravenously, the pharmaceutical composition comprising the chemotherapeutic agent, or the anti-CD20 antibody, can be administered by infusion over a period of about 0.5 to about 10 hours, such as about 2 to about 8 hours, e.g., over a period of about 3 to about 7 hours, such as over a period of about 4 to about 6 hours, or over a period of about 6 hours. In some embodiments, infusion occurs over a period of about 0.5 to about 2.5 hours, over a period of about 0.5 to about 2.0 hours, over a period of about 0.5 to about 1.5 hours, or over a period of about 1.5 hours, depending upon the agent being administered.

In one particular embodiment, the chemotherapeutic agent is administered once by intravenous injection and the IL-2 or variant thereof and, optionally, the anti-CD20 antibody, are administered in a first dose on the same day as the chemotherapeutic agent. Subsequent intermittent therapy using the IL-2 and, optionally, the anti-CD20 antibody, is then performed. Alternatively, the patient can be pretreated with IL-2 or variant thereof, by the delivery of one to five or more doses, such as two or three doses, prior to dosing with the chemotherapeutic agent and, optionally, the anti-CD20 antibody.

Factors influencing the respective amount of the various compositions to be administered include, but are not limited to, the mode of administration, the frequency of administration (i.e., daily, or intermittent administration, such as twice- or thrice-weekly), the particular chemotherapeutic regimen used, the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

See, for example, the treatment protocols disclosed in Gluck et al., Clin. Cancer Res. (2004) 10:2253-2264; copending U.S. Patent Publication 20030185796 and copending U.S. Patent Application No. 60/491,371, the contents of which are herein incorporated by reference in their entireties. The amount of IL-2 (either native-sequence or variant thereof retaining IL-2 biological activity, such as muteins disclosed herein) administered may range between about 0.1 to about 15 mIU/m². See Gluck et al., Clin. Cancer Res. (2004) 10:2253-2264; copending U.S. Patent Publication 20030185796 and copending U.S. Patent Application No. 60/491,371 for recommended doses for IL-2 and rituximab therapy for B-cell lymphomas and CLL. For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. For example, the dosage of the anti-CD20 antibody to administer to a human subject can be from 100 mg/m² to 750 mg/m², generally 200 mg/m² to 500 mg/m², more preferably 300 mg/m² to 400 mg/m², such as 300 . . . 310 . . . 320 . . . 330 . . . 340 . . . 350 . . . 360 . . . 370 . . . 375 . . . 380 . . . 390 . . . 400, and so on, or any integer between the stated ranges, delivered by i.v. infusion. See, Gordon et al., J. Clin. Oncol. (January 2005).

Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration of the antibodies by modifications such as, for example, lipidation.

Appropriate doses and chemotherapeutic compositions for use with the present invention are known in the art. For example, CHOP and the individual constituents thereof, can be administered as described in Mohammad et al., Clin. Cancer Res. (2000) 6:4950; McKelvey et al., Cancer (1976) 38:1484-1493; Armitage et al., J. Clin. Oncol. (1984) 2:898-902; Skeel, R. T., Handbook of Cancer Chemotherapy, 3rd Edition, Little, Brown & Co., 1991:343; and U.S. Pat. Nos. 6,645,983; 6,455,043; 6,593,342, incorporated herein by reference in their entireties. Typical routes of administration are i.p., i.v. or p.o. Regimens may be either daily (qd), as described above, every other day (q2d), etc, such as daily dosing for 8 days (qd×8), q4d×3 (3 doses given on day 1, 5, 9, etc). Typical doses of the CHOP components are as follows: cyclophosphamide, up to 200 mg/kg single dose i.v. or i.p., or 20 mg/kg qd×8 i.v. or i.p.; doxorubicin, up to 6 mg/kg single does or qd4×3 i.v. or i.p.; vincristine, 0.2 to 0.5 mg/kg single dose or qd×8 i.p. or i.v.; prednisone, up to 10 mg/kg/day as a single agent, p.o. Another regimen useful in humans is the following: cyclophosphamide (CTX) 750 mg/m² i.v, D1, doxorubicin (DOX) 50 mg/m² i.v., D1, vincristine (VCR) 1.4 mg/m² i.v., D1 and prednisone (Pred) 100 mg per day p.o., D1-5, with a 21 day cycle.

For CHOP-BLEO, a typical regimen is CTX 750 mg/m² i.v., D1, DOX 50 mg/m² i.v., D1, VCR 2 mg i.v., D1,5, Pred 100 mg per day p.o., D1-5, bleomycin (BLEO) 15 units per day i.v., D1-5, with a cycle of 14 or 21 days (see, e.g., Rodriguezet al., BLOOD (1977) 49:325-333.

For COMLA, a typical regimen is CTX 1500 mg/m² i.v., D1, VCR 1.4 mg/m² i.v., D1,8,15, methotrexate (MTX) 120 mg/m² i.v., D22,29,36,43,50,57,64,71, leucovorin (Leu) 25 mg/m² p.o., D23,30,37,44,51,58,65,72 q6 h×4 doses beginning 24 hr post MTX, cytarabine (ARA-C) 300 mg/m² i.v., D22,29,36,43,50,57,64,71, with a 91 day cycle (see, e.g., Gaynor et al., J. Clin. Onclol. (1985) 3:1596-1604.

For COP a typical regiment is CTX 400-800 mg/m² i.v., D1, VCR 2 mg i.v., D1, Pred 60 mg/m² per day p.o, D1-5, followed by tapering the dose to 40, 20, 10 mg/day, with a 14 day cycle (see, e.g., Luce et al., Cancer (1971) 28:306-317.

For CVP-1 a typical regiment is CTX 400 mg/m² p.o., D1-5, VCR 1.4 mg/m² i.v., D1, Pred 100 mg/m² per day p.o, D1-5, with a 21 day cycle (see, e.g., Bagley et al., Ann. Intern. Med. (1972) 76:227-234.

For DHAP, a typical regimen is cisplatin (CDDP) 100 mg/m² c.i.v. over 24 hours, D1, ARA-C 2 g/mg/m² i.v. over 3 hours, D2, dexamethasone (DEX) 40 mg per day p.o. or i.v., D1-4 for 4 days, with a cycle of 3-4 weeks (see, e.g., Velasquez et al., Blood (1988) 71:117-122).

For ESAP, a typical regimen is methylprednisolone (SOL) 500 mg per day i.v., D1-4, etoposide (VP-16) 40 mg/m² per day i.v., D1-4, ARA-C 2 g/m² i.v., D5 over 2 hours, after completion of CDDP, CDDP 25 mg/m ² per day×4 c.i.v., D1-4 (total dose 100 mg), with a cycle as tolerated (see, e.g., Velasquez et al., Proc. Asco. (1989) 8:256).

For MACOP-B, a typical regimen is MTX 400 mg/m² i.v., weeks 2, 6 and 10, Leu 15 mg p.o. q 6 hr×6 doses, starting 24 hours after MTX, DOX 50 mg/m² i.v., weeks 1, 3, 5, 7, 9, 11, CTX 350 mg/m² i.v., weeks 1, 3, 5, 7, 9, 11, VCR 1.4 mg/m² i.v., weeks 2, 4, 6, 8, 10, 12, BLEO 10 units/m² i.v., weeks 4, 8, 12, Pred 75 mg per day p.o., tapered over the last 15 days (see, e.g., Connors et al., eds. Update on Treatment for Diffuse Large Cell Lymphoma. Wiley & Sons (1986):37-43).

For MIV, a typical regimen is mitoxantrone (DHAD) 10 mg/m² i.v., D1, ifosfamide (IFF) 1500 mg/m² per day i.v., D1-3 with MESNA, VP-16 150 mg/m² per day i.v., D1-3, with a cycle of 21 days (see, e.g., Herbrecht et al., Proc. Asco. (1991) 10:278).

For m-BACOD, a typical regimen is MTX 200 mg/m² i.v., D8,15, Leu 10 mg/m² p.o., D9,16 q 6 h×8 doses starting 24 hours after MTX, DOX 45 mg/m² i.v., D1, CTX 600 mg/m² i.v., D1, VCR 1 mg/m² i.v., D1, DEX 6 mg/m² per day p.o., D1-5, with a cycle of 3 weeks (see, e.g., Shipp et al., Ann. Intern. Med. (1986) 104:757-765.

For PROMACE-CYTABOM, a typical regimen is Pred 60 mg/m² per day p.o., D1-14, DOX 25 mg/m² i.v., D1, CTX 650 mg/m² i.v., D1, VP-16 120 mg/m² i.v., D1, ARA-C 300 mg/m² i.v., D8, BLEO 5 units/m² i.v., D8, VCR 1.4 mg/m² i.v., D8, MTX 120 mg/m² i.v., D8, Leu 25 mg p.o., D9 q 6 h×4 doses starting 24 hours after MTX, with a cycle of 21 days, next cycle beginning on D22 (see, e.g., Fisher et al., Proc. Asco (1984):242 Abstract).

For VACOP-B, a typical regimen is VP-16 50 mg/m² i.v., D1 and 100 mg/m² per day p.o., D2,3 of weeks 3, 7, 11, DOX 50 mg/m² i.v., weeks 1, 3, 5, 7, 9, 11, CTX 350 mg/m² i.v., weeks 1, 5, 9, VCR 1.2 mg/m² i.v., weeks 2, 4, 6, 8, 10, 12, Pred 45 mg/m² p.o., q D×1 week, then q OD×11 weeks, BLEO 10 units/m² i.v., weeks 2, 4, 6, 8, 10, 12 (see, e.g. Connors et al. Proc. Asco. (1990) 9:254).

One of skill in the art can readily determine other appropriate chemotherapeutic doses for these and other regimens. See, for example, Freedman and Nadler, “Non-Hodgkin's Lymphomas” in Cancer Medicine, Vol. 2, Part 6, Holland & Frei (eds.).

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, a subject may receive one or more additional treatment periods comprising IL-2 therapy in combination with anti-CD20 antibody administration, and/or administration of a chemotherapeutic agent. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Materials and Methods:

A. IL-2

The IL-2 formulation used was manufactured by Chiron Corporation of Emeryville, Calif., under the tradename Proleukin®. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed in E. coli, and subsequently purified by diafiltration and cation exchange chromatography as described in U.S. Pat. No. 4,931,543. The IL-2 formulation marketed as Proleukin® is supplied as a sterile, white to off-white preservative-free lyophilized powder in vials containing 1.3 mg of protein (22 MIU).

B. Anti-CD20 Antibody

The anti-CD20 antibody used was Rituxan® (rituximab; IDEC-C2B8; IDEC Pharmaceuticals Corp., San Diego, Calif.) (“R”).

C. CHOP

The constituents of CHOP, (cyclophosphamide, C; Doxorubicin, H; Vincristine, O; and Prednisone, P) used were as follows:

Cytoxan (C, cyclophosphamide)

powder for injection—intravenous—lyophilized 500 mg

Cytoxan Lyophilized, Bristol-Myers Squibb.

Doxorubicin (H)

solution—intravenous—2 mg/ml

Adriamycin, Bedford Laboratories

Vincristine (O)

solution—intravenous—1 mg/ml

Vincristine Sulfate, SICOR Pharmaceuticals Inc.

Prednisone (P)

solution—oral—5 mg/5 ml

Prednisone, Roxane Laboratories Inc.

D. Cell Lines

The human B-cell NHL Daudi cell line was obtained from American Type Culture Collection (Manassas, Va.). Cells were cultured in RPMI supplemented with 10% heat-inactivated fetal bovine serum (FBS, Gibco Life Technologies, Gaithersburg, Md.). Cells were grown as suspension cultures and maintained in a humidified atmosphere at 37° C. and 5% CO₂. Cells were used in exponential growth phase, with viability greater than 98% (assessed using trypan blue exclusion) and determined free of mycoplasma.

E. Mice

Athymic BALB/c nude mice (4-6 weeks of age) were obtained from Charles River Laboratories, Inc. (Wilmington, Mass.) and acclimated for 1 week prior to the start of studies. Mice received sterile rodent chow and water ad libitum and were housed in sterile filter-top cages with a 12-hour light/dark cycle. All in vivo studies were conducted in compliance with the Institutional Animal Care and Use Committee and the Guidelines for the Care and Treatment of Laboratory Animals.

F. In Vivo Efficacy Studies and Assessment of Tumor Inhibition and Responses

Passaged, clonally derived Daudi cells (5×10⁶ cells/mouse) were reconstituted with 50% Matrigel (BD Biosciences) in a total volume of 0.1 ml and implanted subcutaneously into the right flank of irradiated (3Gy) BALB/c nude mice. Treatments were initiated when the mean tumor volume was 150-300 mm³. Mice were randomly assigned typically into 10 mice per group. For groups that were given CHOP, this treatment consited of 40 mg/kg cyclophosphamide, i.v.; 3.3 mg/kg doxirubicin, i.v.; 0.5 mg/kg vincristine, i.v. on day 1; and 0.2 mg/kg prednisone, days 1-5, p.o. (Mohammad et al. (2003) Mol. Cancer Ther. 2:1361-8). Tumor volume is measured using calipers. Caliper measurements of tumors were converted into tumor volume (mm³) using the formula: ½ (length (mm)×[width (mm)]²). Tumor growth inhibition (TGI) was calculated as [1−(mean tumor volume of treated group/mean tumor volume of control group)×100]. Responses were defined as either a complete response (CR, no measurable tumor), partial response (PR, 50-99% tumor volume reduction compared to tumor volume for each animal at treatment initiation), Minor Response (MR, maximal tumor inhibition of 25 to 50% of the initial tumor volume on day 1), Stable Disease (SD, tumor growth is +/−25% of the initial tumor volume). Tumor growth delay (TGD) analysis was calculated as: [(number of days for a treated group to reach a mean tumor volume of 1000 mm³)−(number of days for the control group to reach a mean tumor volume of 1000 mm³)]. Percent conditional survival is the percent of mice in each group in which the tumor volume has not reached 1000 mm³.

G. Statistical Analysis

Multiple comparisons were performed using one-way analysis of variance (ANOVA); post-test to compare different treatment means was done using Student-Newman Keuls test (SigmaStat). Tumor growth delay for each animal (time to 1000 mm³) was used as the endpoint for conditional survival, and significance between treatments was analysed using a log rank test (Prism). Mice that had no visible tumors at necropsy upon termination of the study were considered censored in this analysis. Differences were considered statistically significant at p<0.05. Synergistic responses were defined when the ratio of expected % inhibition of each therapy (% T/C treatment 1×% T/C treatment 2) divided by observed % T/C of the combination treatment group was >1 (Yokoyama, Y. et al. Cancer Res. (2000) 60:2190-2196).

### Example 1

### Evaluation of various IL-2/CHOP/Rituximab Regimens for Treating Human B-Cell Lymphoma

Combination IL-2 (Proleukin®), Rituximab and CHOP administration was evaluated in the Daudi xenograft model of human B-cell lymphoma as follows. See, e.g., Hudson et al., Leukemia (1998) 12:2029-2033 for a description of the Daudi xenograft model. The Daudi/BALB/c nude model expresses high levels of CD20 and is associated with a less aggressive/low grade disease profile. Furthermore, NK cells cannot lyse Daudi tumor cells in the absence of activation by cytokines such as IL-2. See, e.g., Damle et al., J. Immunol. (1987) 138:1779-1785.

120 athymic nude BALB/c mice were acclimated for 1 week to the inoculation of Daudi human B-cell lines. Daudi cells (5×10⁶ cells/mouse) were implanted s.c. (right flank) into irradiated young nude mice (3Gy for approximately 3.2 minutes) with 50% matrigel at a volume of 0.1 ml and grown as subcutaneous tumors until tumor volume reached 110 mm³. This was designated study day 1.

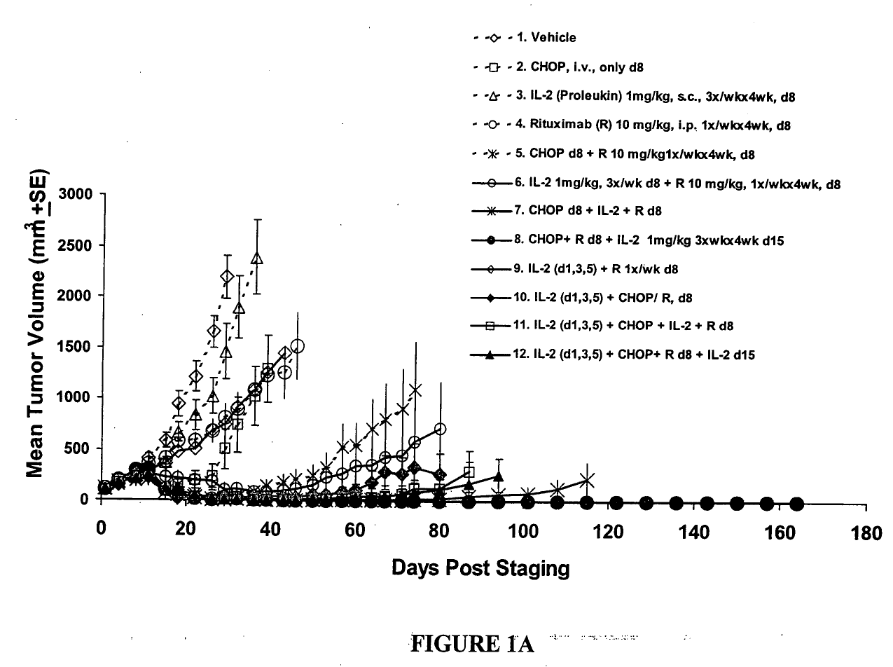

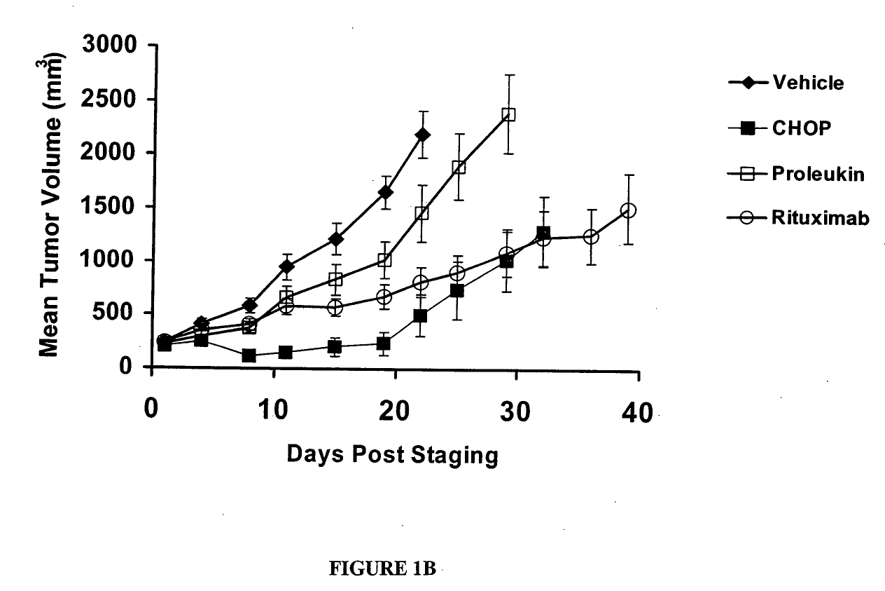

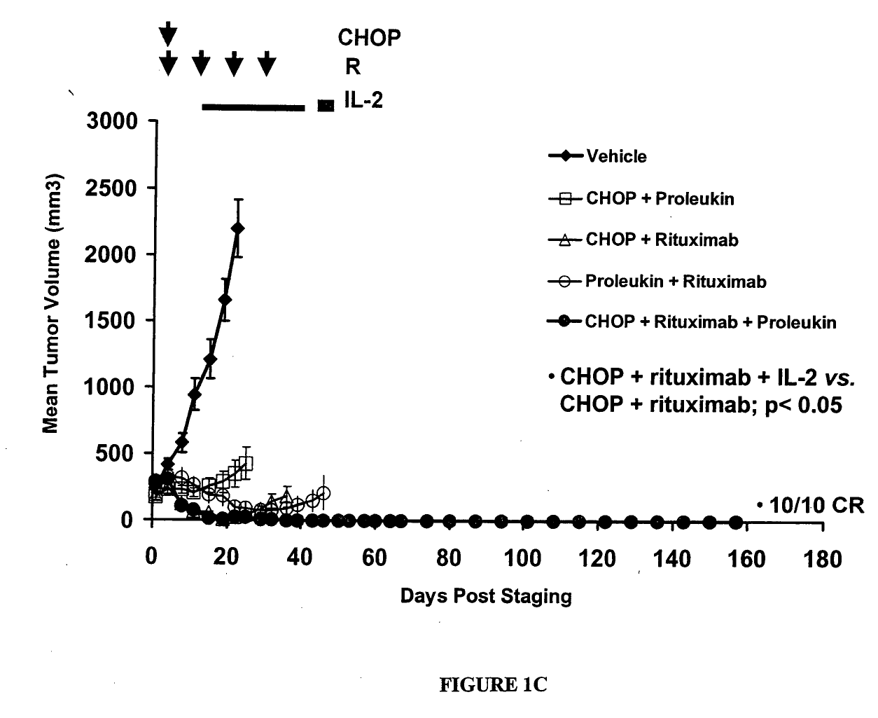

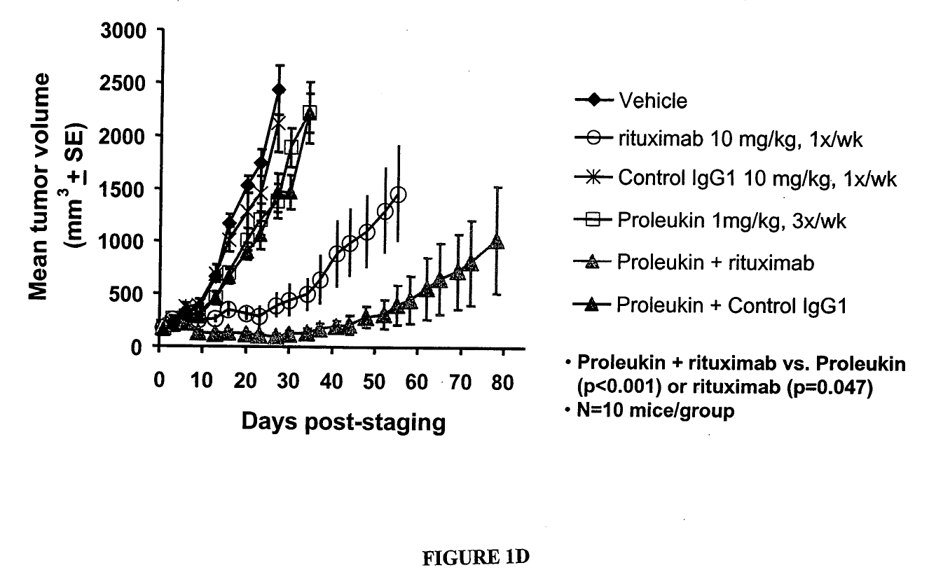

CHOP and Rituximab administration was modeled on known clinical dosage regimens (see, Mohammad et al., Clin. Cancer Res. (2000) 6:4950) and treatment commenced when tumors were established (150-200 mm³). Treatment groups comprised either CHOP alone (C, 40 mg/kg, i.v; H, 3.3 mg/kg, i.v; O, 0.5 mg/kg, i.v all administered on Day 8 and P, 0.2 mg/kg, days 8-12) or a 4 week cycle of rituximab (R) alone (10 mg/kg, once weekly, i.p) starting day 8 or CHOP in combination with a 4 week cycle of rituximab. Proleukin® treatment (1 mg/kg; s.c., thrice weekly) commenced either concomitantly (on day 8) or one week later (day 15) for a total of 4 weeks. See, Table 1 and FIGS. 1A-1C. Tumor volumes and body weights were measured two times per week. Clinical observations were noted.

TABLE 1

| Day 1-7 | | Day 8 |
|---|---|---|
| 1. | Untreated | Vehicle sc. 0.2 ml 3x/wk |
| 2. | Untreated | CHOP iv., only day 8 |
| 3. | Untreated | IL-2 (Proleukin ®) 1 mg/kg sc. |
| | | 3x/wk × 4 wk, day 8 |
| 4. | Untreated | Rituximab 10 mg/kg ip. 1x/wk × 4 wk, day 8 |
| 5. | Untreated | CHOP iv. day 8 + Rituximab 10 |
| | | mg/kg ip. 1x/wk × 4 wk, day 8 |
| 6. | Untreated | IL-2 1 mg/kg sc. 3x/wk day 8 + Rituximab |
| | | 10 mg/kg ip. 1x/wk × 4 wk, day 8 |
| 7. | Untreated | CHOP iv day 8 + IL-2 1 mg/kg sc. 3x/wk × |
| | | 4 wk day 8 + Rituximab 10 mg/kg ip. 1x/wk × 4 |
| | | wk day 8 |
| 8. | Untreated | CHOP iv. day 8 + Rituximab 10 mg/kg 1x/wk × |
| | | 4 wk, day 8 + IL-2 sc. 3x/wk × 4wk/, day 15 |
| 9. | IL-2 (day 1, 3, 5) | Rituximab 10 mg/kg ip. |
| | | 1x/wk × 4 wk, day 8 |
| 10. | IL-2 (day 1, 3, 5) | CHOP iv. day 8 + Rituximab 10 mg/kg |
| | | ip. 1x/wk × 4 wk, day 8 |
| 11. | IL-2 (day 1, 3, 5) | CHOP iv. day 8 + IL-2 1 mg/kg sc. 3x/wk × |
| | | 4 wk, day 8 + Rituximab 10 mg/kg ip.6 |
| | | 1x/wk × 4 wk, day 8 |
| 12. | IL-2 (day 1, 3, 5) | CHOP iv. day 8 + Rituximab 10 mg/kg ip. |
| | | 1x/wk × 4 wk, day 8 + IL-2 1 mg/kg sc. |
| | | 3x/wk × 4 wk, day 15 |

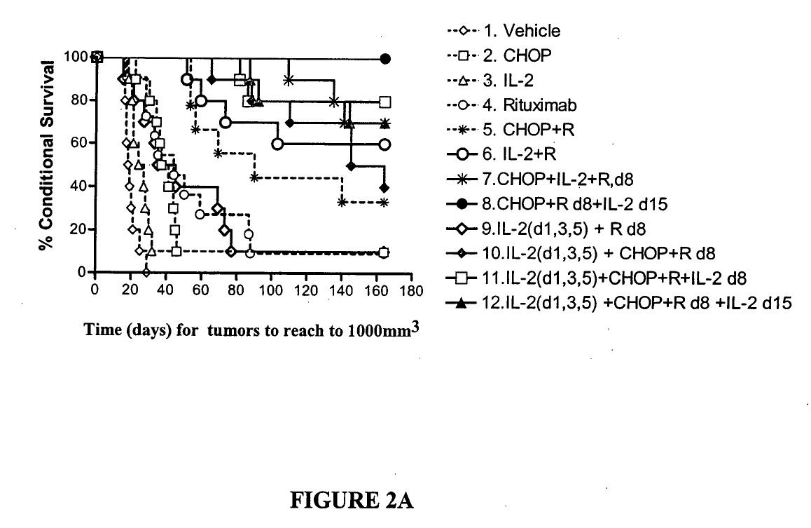

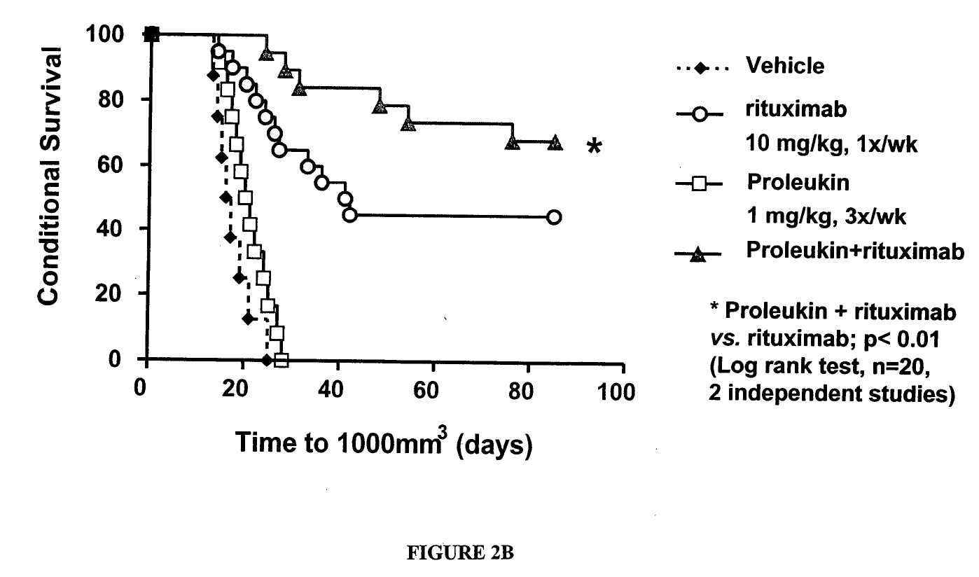

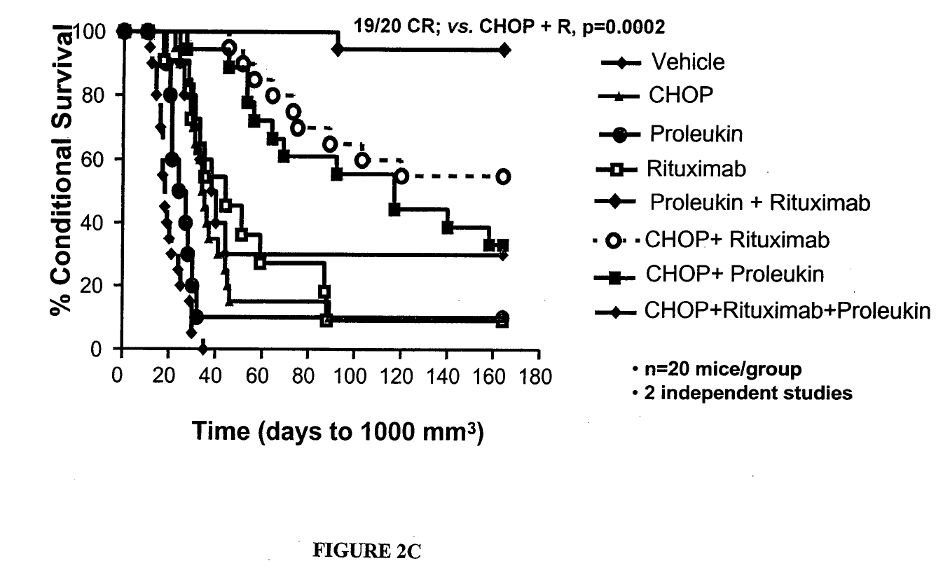

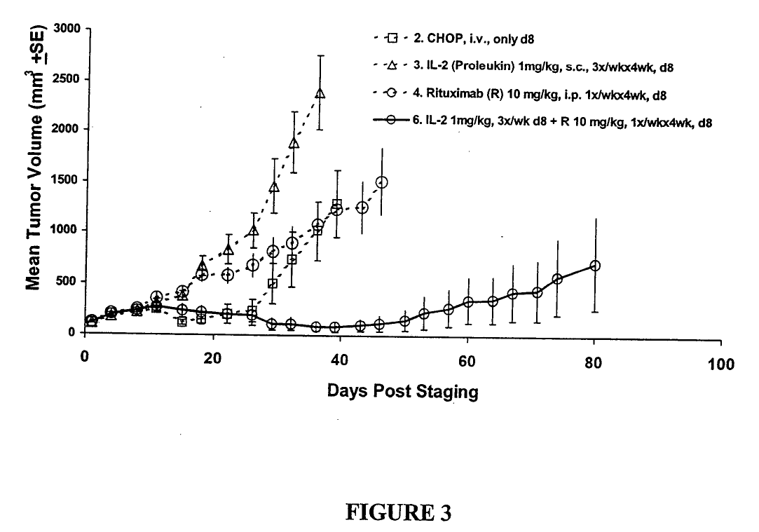

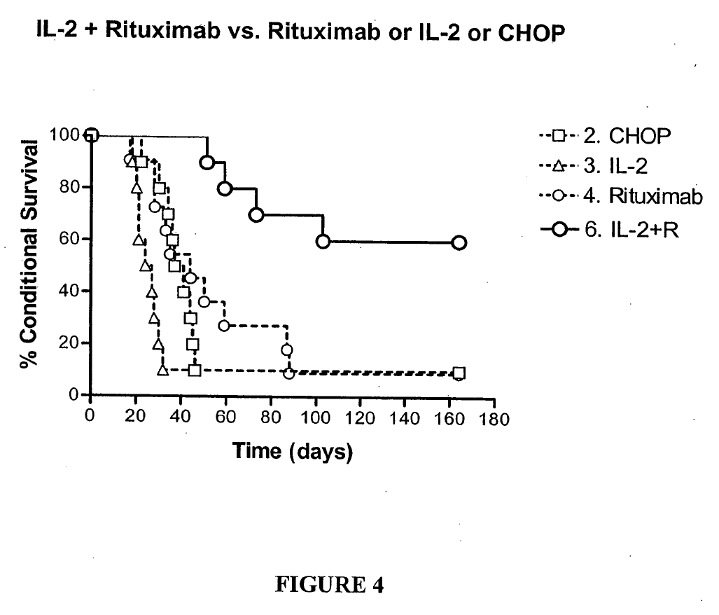

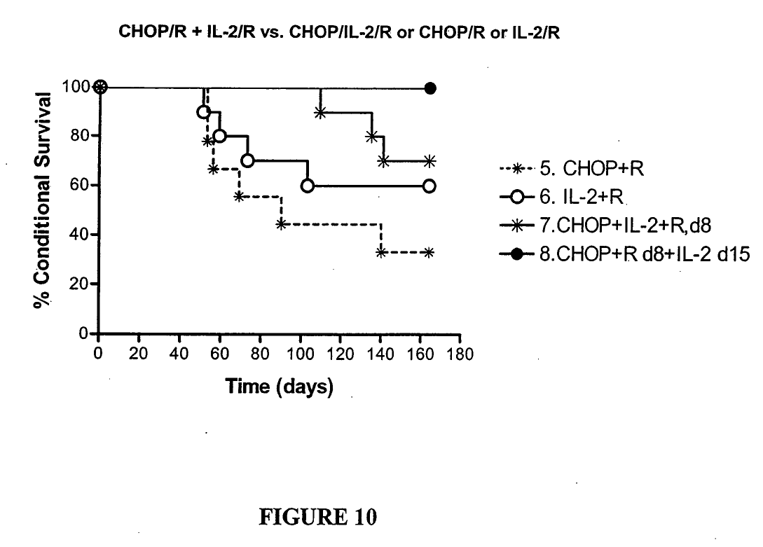

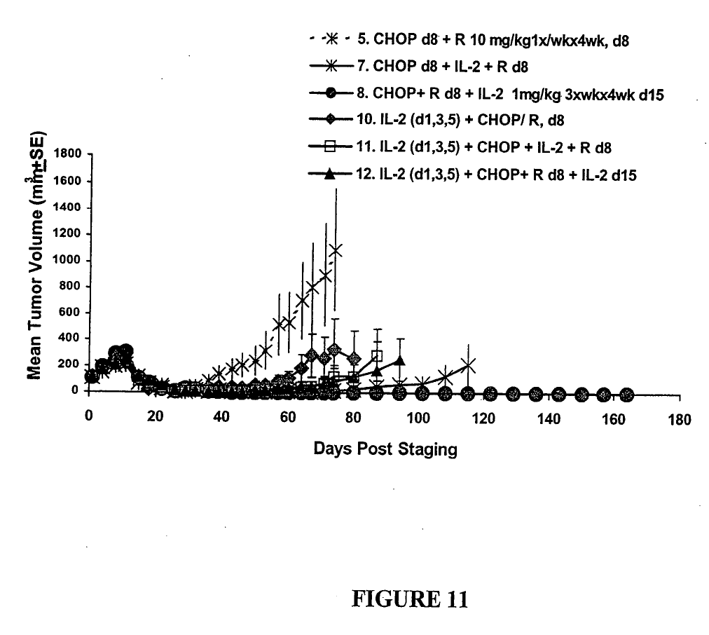

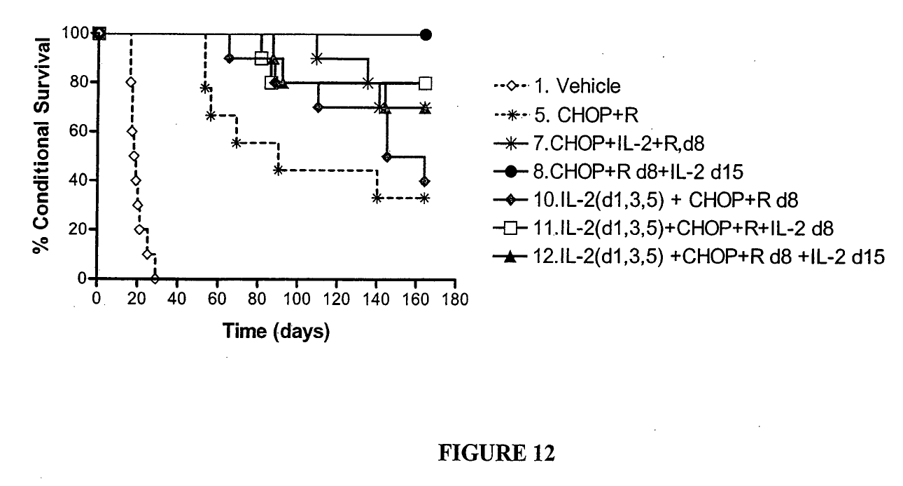

Results are shown in FIGS. 1-13 and Tables 2 and 3. In particular, in the Daudi xenograft model, single agents IL-2, CHOP, rituximab (R) and combined treatments of IL-2/rituximab, CHOP/rituximab, and IL-2/rituximab at dose regimens tested significantly inhibited tumor growth compared to vehicle treatments (p<0.001, ANOVA day 29) (FIGS. 1-4; Tables 2, and 3). IL-2/rituximab significantly inhibited tumor growth and delayed time to reach a tumor volume of 1000 mm³ (tumor growth delay, TGD) compared to single test agents IL-2, rituximab (p<0.01, Log Rank, TGD and p<0.05, ANOVA day 29) (FIG. 4, Table 3; IL-2/group 3 vs. IL-2 and rituximab/group 6). Combined IL-2 and rituximab therapy demonstrated significantly improved efficacy compared to CHOP alone in terms of delaying time to tumor progression and the number of evaluable responses (IL-2/R=6CR; CHOP=1CR).

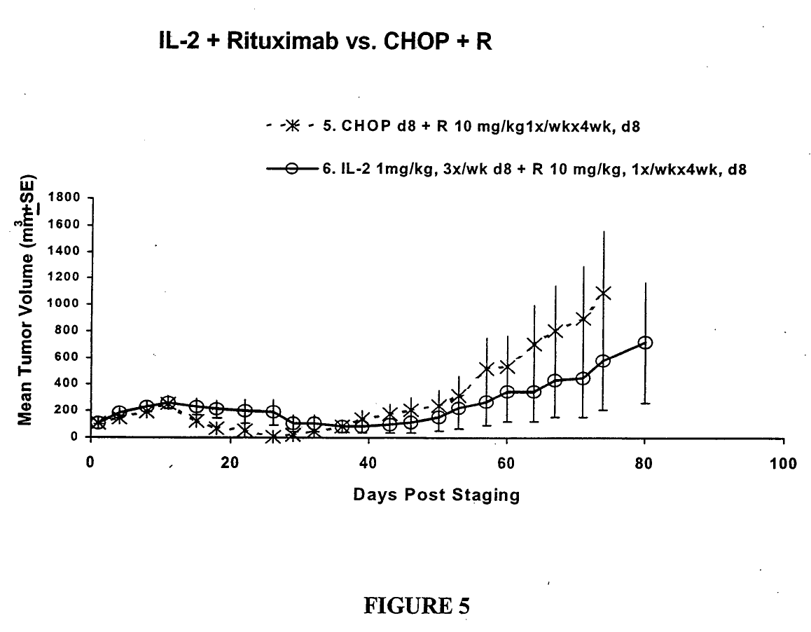

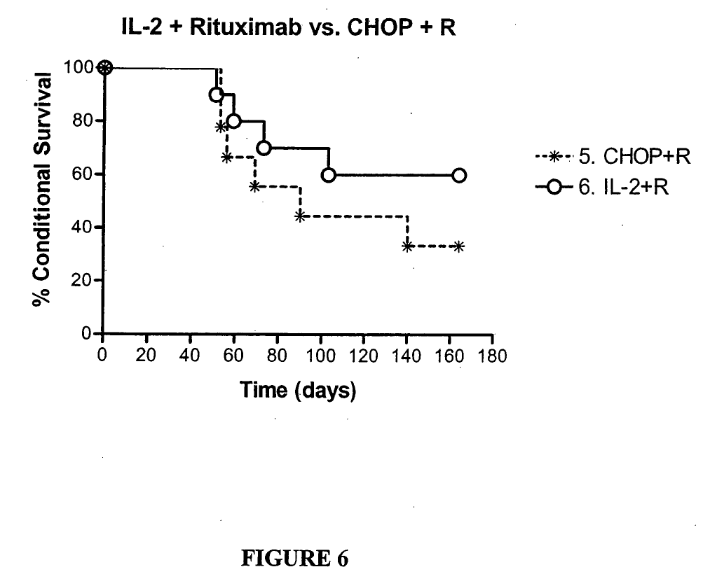

Moreover, combinations of IL-2/R demonstrated equivalent efficacy compared to CHOP/R therapy in tumor growth inhibition (TGI) and TGD to 1000 mm³ analyses (p=0.274 Log Rank TGD to 1000 mm³ and p=0.579 ANOVA day 29) (FIGS. 5 and 6).

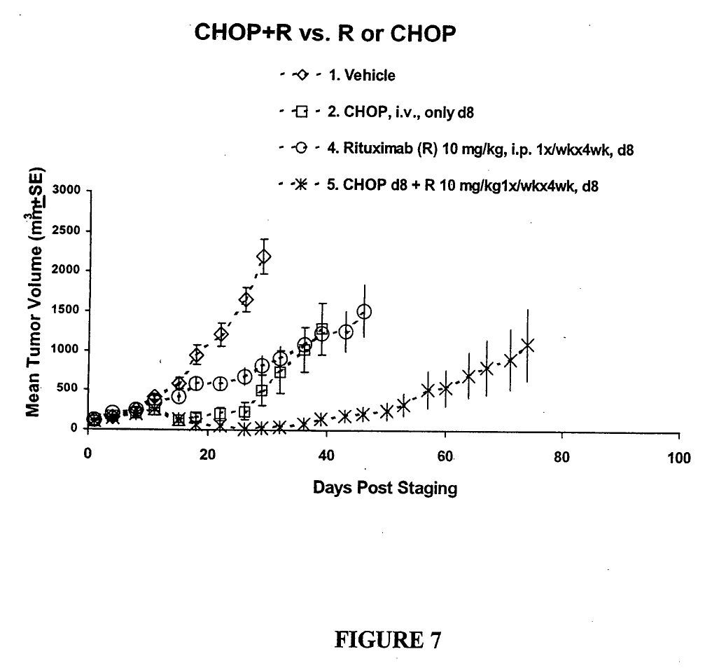

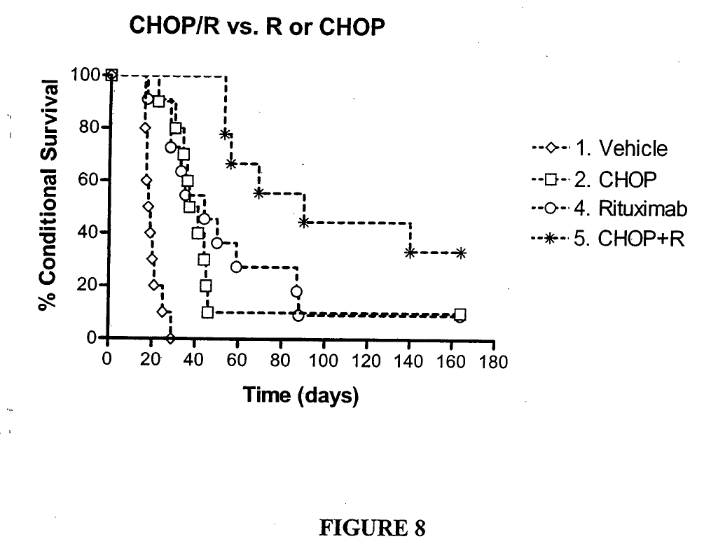

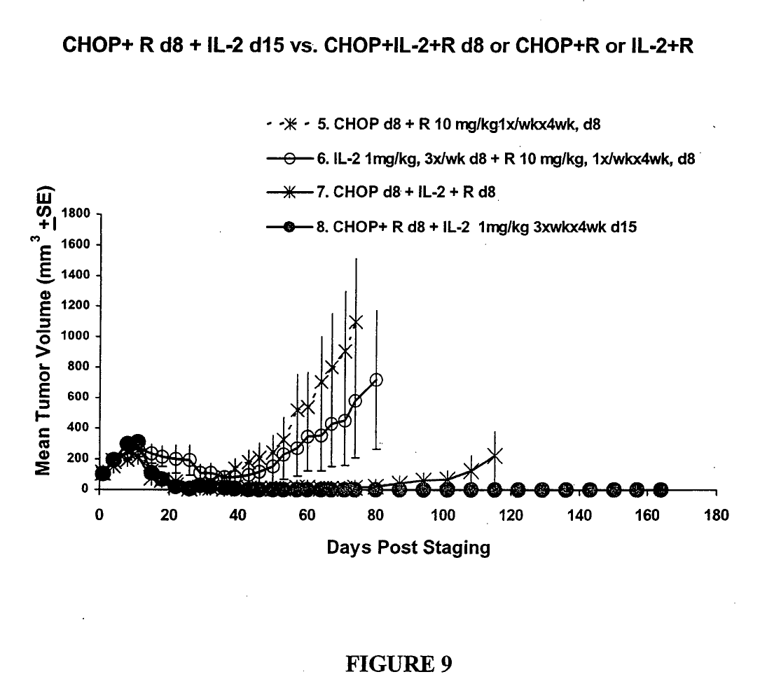

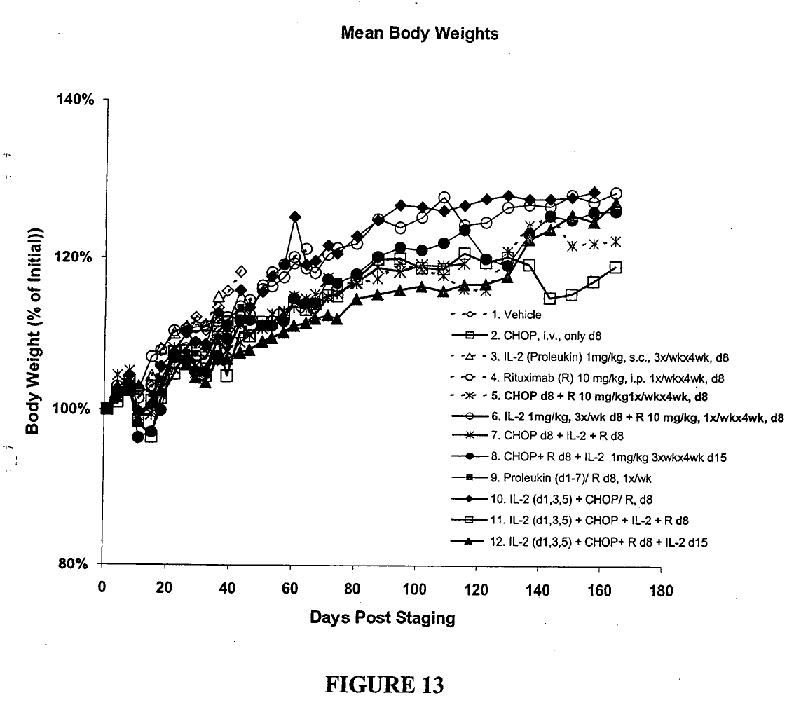

CHOP/R significantly inhibited tumor growth (99% tumor growth inhibition, TGI; 5/10 CR) and was superior to either R (63% TGI, 1 CR) or CHOP (77% TGI, 1 CR) alone (FIGS. 7 and 8). Notably, the addition of IL-2 to CHOP/R (commenced either on D8 or D15, see experimental design) induced substantial tumor regression with 7/10 and 9/10 CR respectively and was superior to CHOP/R (p<0.05) (FIGS. 9-12) Moreover, the addition of IL-2 to CHOP/R significantly increased the time to progression (>164 days) compared to CHOP/R alone (90 days) (FIGS. 9-12). All doses of test agents were well tolerated (FIG. 13). In summary, CHOP/R followed by IL-2/R is safe, efficacious and is predicted to add benefit in delaying time to progression compared to the use of CHOP or CHOP/R alone. These findings are expected to apply to combinations of chemotherapies with other antibodies and/or mediated via other immunostimulatory cytokines/agents.

TABLE 2

| | | TGI vs. | | | | |
|---|---|---|---|---|---|---|
| | | vehicle | | | | |
| | | (day | | | | |
| | TGI | 29) P | CR/PR | CR/PR | % max BW. change | Clinical |
| Treatment n = 10 | day 29 | Value¹ | day 29 | day 164 | (day, range) | Observation² |
| 1. Vehicle | | | | | 12(+7 to +18) day 29 | BAR |
| 2. CHOP d8 | 77.17% | <0.001 | 1/0 | 1/0 | 7(0 to +9) day 32 | BAR |
| 3. IL-2 d8 | 33.52% | <0.001 | 0/0 | 1/0 | 12(+8 to +14) day 39 | BAR |
| 4. R d8 | 63.03% | <0.001 | 0/0 | 1/0 | 14(+10 to +19) day | BAR |
| | | | | | 46 | |
| 5. CHOP + R d8 | 98.95% | <0.001 | 5/2 | 5/0 | 17(+10 to +21) day | BAR |
| | | | | | 71 | |
| 6. IL-2 + R d8 | 95.09% | <0.001 | 4/3 | 6/0 | 22(+12 to +35) day | BAR |
| | | | | | 80 | |
| 7. CHOP + IL-2 + R | 99.51% | <0.001 | 8/2 | 7/0 | 18(+7 to +26) day 94 | BAR |
| d8 | | | | | | |
| 8. CHOP + R d8 + IL- | 98.76% | <0.001 | 5/2 | 9/0 | 21(+13 to +28) day | BAR |
| 2 d15 | | | | | 94 | |
| 9. IL-2(d1, 3, 5) + R | 66.31% | <0.001 | 0/1 | 1/0 | 13(+5 to +18) day 43 | BAR |
| d8 | | | | | | |
| 10. IL- | 99.22% | <0.001 | 8/1 | 4/0 | 23(+15 to +29) day | BAR |
| 2(d1, 3, 5) + CHOP + R | | | | | 80 | |
| d8 | | | | | | |
| 11. IL- | 99.70% | <0.001 | 9/1 | 8/0 | 19(+7 to +26) day 80 | BAR |
| 2(d1, 3, 5) + CHOP + IL- | | | | | | |
| 2 + R d8 | | | | | | |
| 12. IL- | 99.54% | <0.001 | 8/2 | 7/0 | 12(+2 to +19) day 71 | BAR |
| 2(d1, 3, 5) + CHOP + R | | | | | | |
| d8 + IL-2 d15 | | | | | | |

¹ANOVA/Student-Newman-Keul's test
²BAR = bright, alert, responsive

TABLE 3

| | median | |
|---|---|---|
| | conditional | p value |
| | survival | (Logrank) |
| Group | (days to | vs |
| information/treatment | 1000 mm3) | vehicle |
| 1. Vehicle | 19 | |
| 2. CHOP | 39 | <0.0001 |
| 3. IL-2 | 25 | 0.0158 |
| 4. R | 44 | <0.0001 |
| 5. CHOP + R | 90 | <0.0001 |
| 6. IL-2 + R | >164 | <0.0001 |
| 7. CHOP + IL-2 + R, d8 | >164 | <0.0001 |
| 8. CHOP + R d8 + IL-2 d15 | >164 | <0.0001 |
| 9. IL-2(d1, 3, 5) + R d8 | 40 | <0.0001 |
| 10. IL-2(d1, 3, 5) + CHOP + R d8 | >164 | <0.0001 |
| 11. IL-2(d1, 3, 5) + CHOP + IL-2 + R d8 | >164 | <0.0001 |
| 12. IL-2(d1, 3, 5) + CHOP + R d8 + IL2 d15 | >164 | <0.0001 |

### Example 2

### Combination Therapy Using IL-2. CHOP and CHOP Constituents

Combination therapy using IL-2 (Proleukin®) with CHOP and IL-2 with each of the individual CHOP constituents, (cyclophosphamide, C; Doxorubicin, H; Vincristine, O; and Prednisone, P) was evaluated in the Daudi xenograft model of human B-cell lymphoma described above.

Athymic nude BALB/c mice (10 mice/group) were acclimated and irradiated mice were implanted with Daudi cells as described above. Treatment started when tumors were approximately 140 mm³. This was designated study day 1.

CHOP administration was modeled on known clinical dosage regimens (see, Mohammad et al., Clin. Cancer Res. (2000) 6:4950). Treatment groups were as follows:

Cyclophosphamide (C, cytoxan), 40 mg/kg, i.v, day 1;

Doxorubicin (H, adriamycin), 3.3 mg/kg, i.v, day 1;

Vincristine (O), 0.5 mg/kg, i.v, day 1;

Prednisone (P), 0.2 mg/kg p.o, days 1-5;

CHOP (C, 40 mg/kg, i.v; H, 3.3 mg/kg, i.v; O, 0.5 mg/kg, i.v and P, 0.2 mg/kg, p.o, all administered on day 1)

Proleukin® (IL-2), 1 mg/kg; s.c., thrice weekly for 4 weeks, total 12 doses;

Proleukin® (IL-2)+individual chemotherapeutics (as dosed individually, started on day 1);

Proleukin® (IL-2)+CHOP (similar dosing regimens to single agents, started on day 1).

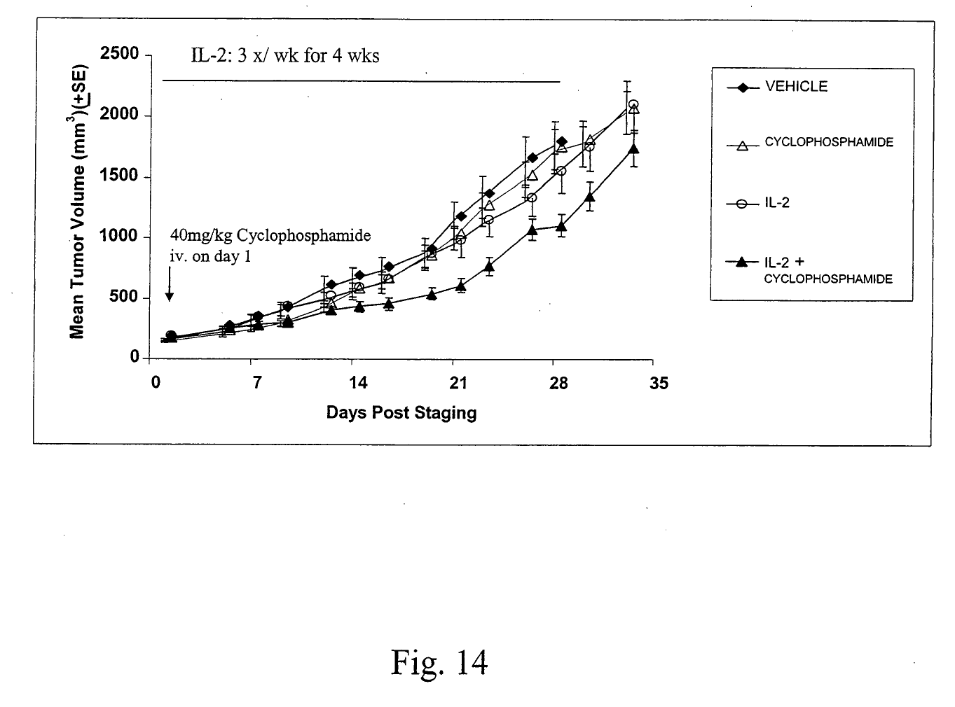

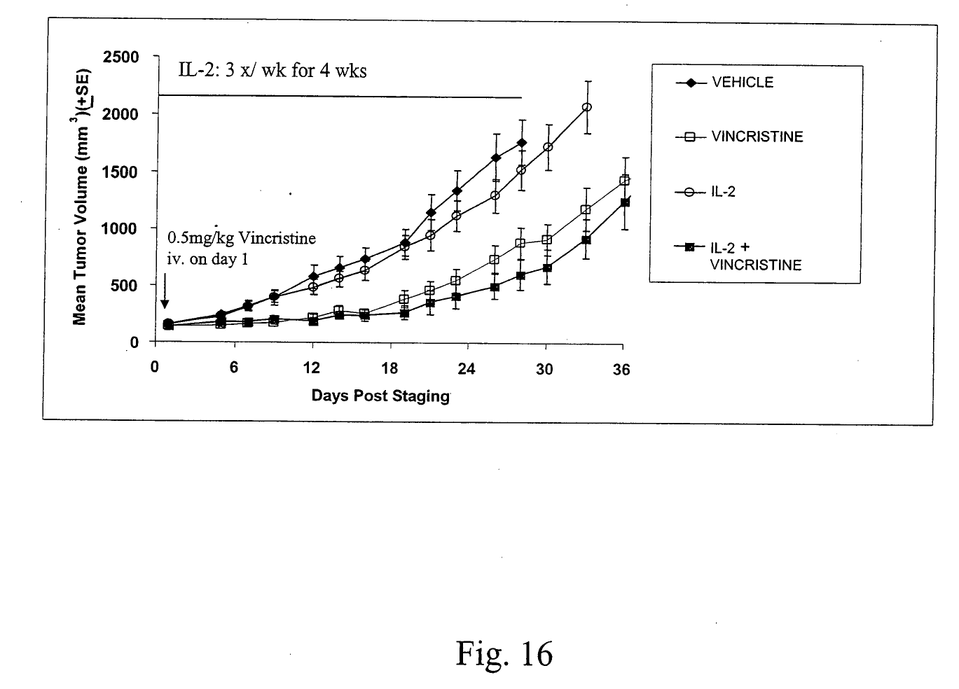

All single agent IL-2, chemotherapeutic agents or combined with either cyclophosphamide, doxorubicin, vincristine or prednisone at doses tested were well tolerated. Single agent IL-2 displayed marginal tumor growth inhibition in this model (14% vs. vehicle, p=0.37). Greater than additive effects were observed with IL-2+cyclophosphamide (FIG. 14 and Table 4) and IL-2+vincristine (FIG. 16 and Table 6) based on tumor growth inhibition, responses, and growth delay analyses.

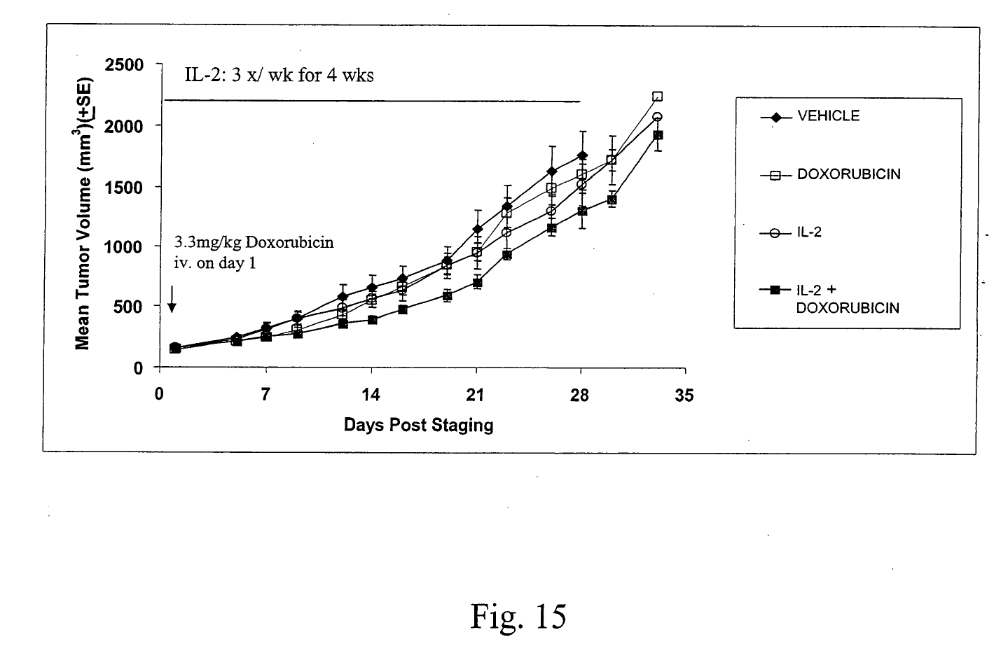

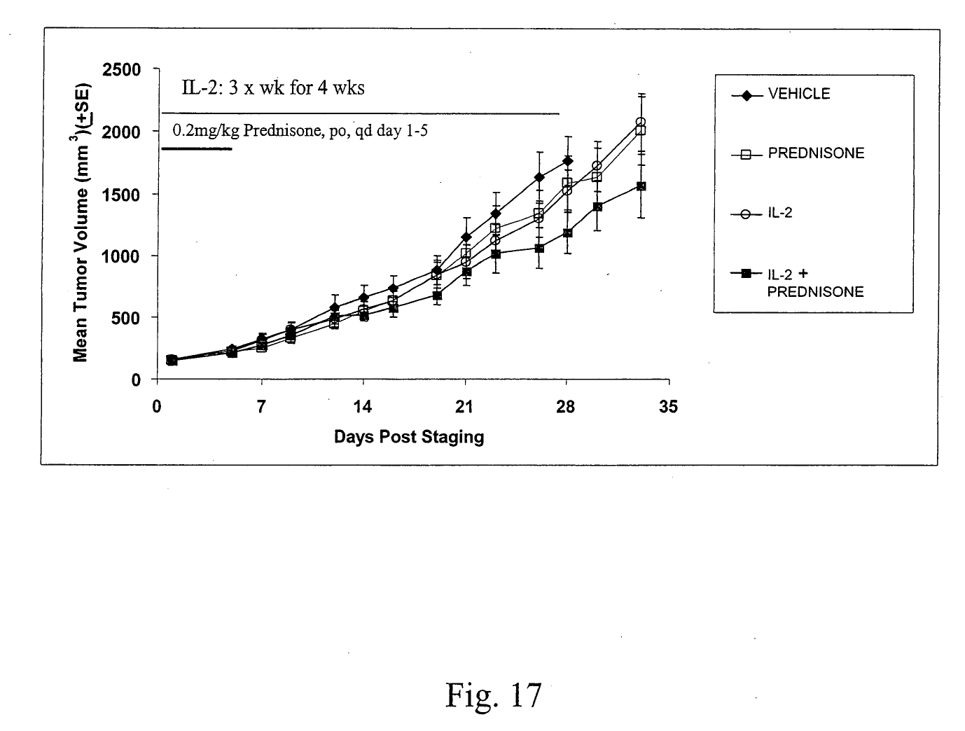

Combinations of IL-2+doxorubicin (FIG. 15 and Table 5) and IL-2+Prednisone (FIG. 17 and Table 7) demonstrated additive responses. IL-2+prednisone demonstrated 21% and 24% inhibition compared to single agent Proleukin® or prednisone, respectively. The results of IL-2+prednisone indicate that combining an immunosuppressive agent such as prednisone, with the dosage schedule used, may not abrogate efficacy.

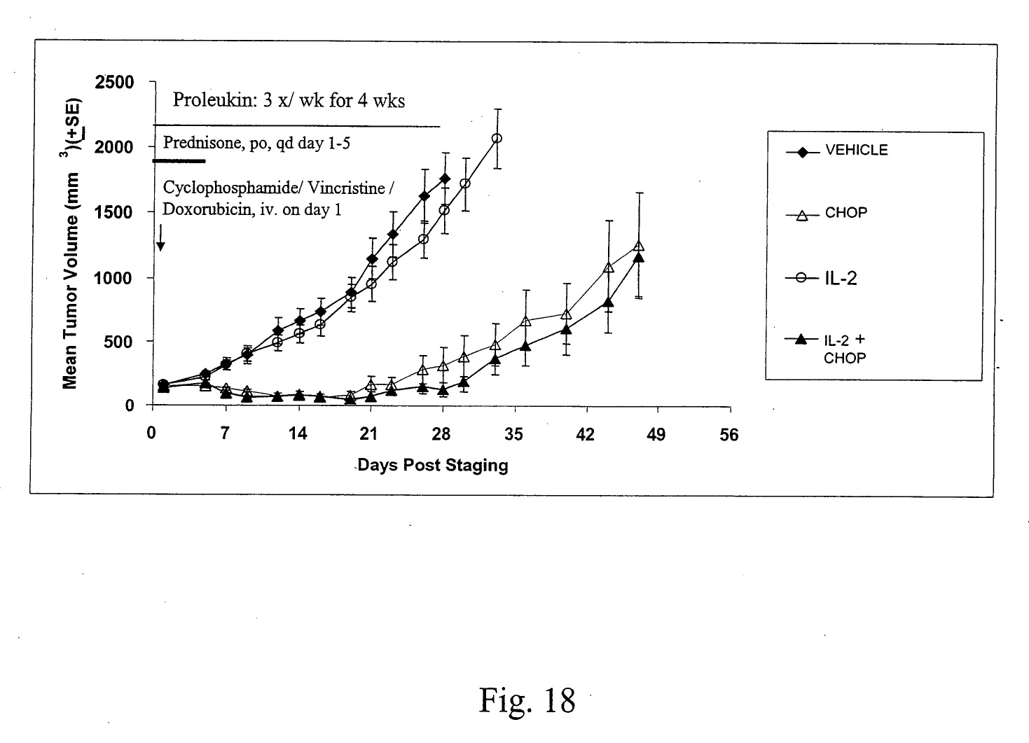

Finally, although IL-2+the individual CHOP constituents were efficacious, combination of IL-2+CHOP demonstrated superior efficacy compared to the single agents alone (FIG. 18 and Table 8) based on extent of tumor growth inhibition, responses and growth delay analyses.

TABLE 4

| | TGI vs. | | | Group Mean | |
|---|---|---|---|---|---|
| | Vehicle (P | TGI vs. IL-2 | TGI vs. | Days of | CR/PR |
| Treatment | value) on day | (P value) on | Cyclophosphamide (P | Delay to | on day |
| (mg/kg) | 28 | day 28 | value) on day 28 | 1000 mm³ | 28 |
| Vehicle | | | | | 0/0 |
| Cyclophosphamide | 2% (0.875) | | | 1 | 0/0 |
| IL-2 | 14% (0.367) | | | 2 | 0/0 |
| IL-2 + Cyclophosphamide | 39% (0.018) | 29% (0.115) | 37% (0.019) | 7 | 0/0 |

TABLE 5

| | TGI vs. Vehicle | | | Group Mean | CR/PR |
|---|---|---|---|---|---|
| Treatment | (P value) on | TGI vs. IL-2 (P | TGI vs. Doxorubicin | Days of Delay | on day |
| (mg/kg) | day 28 | value) on day 28 | (P value) on day 28 | to 1000 mm³ | 28 |
| Vehicle | | | | | 0/0 |
| Doxorubicin | 9% (0.516) | | | 2 | 0/0 |
| IL-2 | 14% (0.367) | | | 2 | 0/0 |
| IL-2 + Doxorubicin | 26% (0.082) | 14% (0.363) | 19% (0.127) | 5 | 0/0 |

TABLE 6

| | TGI vs. Vehicle | | | Group Mean | CR/PR |
|---|---|---|---|---|---|
| Treatment | (P value) on | TGI vs. IL-2 (P | TGI vs. Vincristine (P | Days of Delay | on day |
| (mg/kg) | day 28 | value) on day 28 | value) on day 28 | to 1000 mm³ | 28 |
| Vehicle | | | | | 0/0 |
| Vincristine | 50% (0.002) | | | 12 | 0/0 |
| IL-2 | 14% (0.367) | | | 2 | 0/0 |
| IL-2 + Vincristine | 66% (<0.001) | 60% (p < 0.001) | 32% (0.156) | 15 | 1/0 |

TABLE 7

| | TGI vs. Vehicle | | | Group Mean | CR/PR |
|---|---|---|---|---|---|
| Treatment | (P value) on | TGI vs. IL-2 (P | TGI vs. Prednisone (P | Days of Delay | on day |
| (mg/kg) | day 28 | value) on day 28 | value) on day 28 | to 1000 mm³ | 28 |
| Vehicle | | | | | 0/0 |
| Prednisone | 10% (0.547) | | | 2 | 1/0 |
| IL-2 | 14% (0.367) | | | 2 | 0/0 |
| IL-2 + Prednisone | 32% (0.044) | 21% (0.204) | 24% (0.190) | 5 | 1/0 |

TABLE 8

| | TGI vs. Vehicle | | | Group Mean | CR/PR |
|---|---|---|---|---|---|
| Treatment | (P value) on | TGI vs. IL-2 (P | TGI vs. CHOP (P | Days of Delay | on day |
| (mg/kg) | day 28 | value) on day 28 | value) on day 28 | to 1000 mm³ | 28 |
| Vehicle | | | | | 0/0 |
| CHOP | 82% (<0.001) | | | 24 | 2/1 |
| IL-2 | 14% (0.367) | | | 2 | 0/0 |
| IL-2 + CHOP | 93% (<0.001) | 92% (<0.001) | 60% (0.623) | 26 | 4/1 |

Table 9 shows additional data from 1 or 2 independent studies.

TABLE 9

| | | | | Median | |
|---|---|---|---|---|---|
| | | | | Tumor | |
| | | | Tumor Growth | Growth | Response |
| | | | Inhibition(day, p | delay, | rate |
| Treatment | Dose | Route/schedule | value) | days | (CR/PR) |
| IL-2 | 1 mg/kg | s.c., day 1, 3, 5, 8, | 14% (day 28, | 2 | 0/0 |
| | | 10, 12 | p = 0.367) | | |
| Cyclophosphamide | 40 mg/kg | i.v., day 1 | 2% (day 28, | 1 | 0/0 |
| | | | p = 0.875) | | |
| Doxorubicin | 3.3 mg/kg | i.v., day 1 | 9% (day 28, | 2 | 0/0 |
| | | | p = 0.516) | | |
| Vincristine | 0.5 mg/kg | i.v., day 1 | 50% (day 28, | 12 | 0/0 |
| | | | p = 0.002) | | |
| Prednisone | 0.2 mg/kg | p.o., day 1-5 | 10% (day 28, | 2 | 1/0 |
| | | | p = 0.547) | | |
| CHOP | | i.v., day 1 | 82% (day 28, | 24 | 2/1 |
| | | | p < 0.001) | | |
| IL-2 + cyclophosphamide | 1 mg/kg + 40 mg/kg | IL-2 s.c., day 1, 3, 5, | 39% (day 28, | 7 | 0/0 |
| | | 8, 10, 12 + C i.v., | p = 0.018) | | |
| | | day 1 | | | |
| IL-2 + doxorubicin | 1 mg/kg + 3.3 mg/kg | IL-2 s.c., day 1, 3, 5, | 26% (day 28, | 5 | 0/0 |
| | | 8, 10, 12 + H i.v., | p = 0.082) | | |
| | | day 1 | | | |
| IL-2 + vincristine | 1 mg/kg + 0.5 mg/kg | IL-2 s.c., day 1, 3, 5, | 66% (day 28, | 15 | 1/0 |
| | | 8, 10, 12 + O i.v., | p < 0.001) | | |
| | | day 1 | | | |
| IL-2 + prednisone | 1 mg/kg + 0.2 mg/kg | IL-2 s.c., day 1, 3, 5, | 32% (day 28, | 5 | 1/0 |
| | | 8, 10, 12 + P p.o., | p = 0.044) | | |
| | | day 1-5 | | | |
| IL-2 + CHOP | 1 mg/kg + | IL-2 s.c., day 1, 3, 5, | 93% (day 28, | 26 | 4/1 |
| | | 8, 10, 12 + CHOP | p < 0.001) | | |
| | | i.v., day 1 | | | |

n = 10-20 mice/group from 1 or 2 independent studies;

### Example 3

### Analysis of Cell Populations in Blood and Spleen Following Therapy

To elucidate the mechanistic basis for synergy, the pharmacodynamic responses of key immune effector cell populations after CHOP-R/IL-2 therapy were examined. Treatment started when tumors were approximately 300 mm³. This was designated study day 1. Daudi tumor-bearing BALB/c nude mice were treated with CHOP or CHOP-R on day 1. Immune reconstitution with IL-2/R therapy commenced on day 8. Blood and spleens were collected at various times after indicated treatments from 5 mice per group. Whole blood (100 μl) was transferred to FACS/TruCount tubes (BD BioSciences) and kept on ice. Isolated splenocyte preparations were suspended at a density of 5×10⁶/ml; 5×10⁵ cells per sample were treated and stained with antibodies in 96-well plates as follows. Samples were treated with 0.5 μg mouse Fc block (anti-mouse CD16/CD32; BD BioSciences), and incubated on ice 20 min. Fluorochrome-conjugated antibodies, as indicated below, were added to samples and incubated on ice for 20 min protected from light. Blood samples were vortexed while adding 2 ml of 1×FACS Lysing Solution (BD BioSciences), followed by 10 min incubation at room temperature, and then centrifuged at 1250 rpm. All samples were washed twice, suspended in PBS+2% FBS, and stored at 4° C. prior to sample acquisition on BD FACSCalibur, and subsequent analysis by CellQuest Pro software. Absolute numbers of cells were determined relative to TruCount bead reference. Total lymphocytes (CD45, BD BioSciences), monocytes (F4/80; CalTag; Burlingame Calif.), and NK cells (DX5, BD BioSciences) were identified with respective antibodies. Lymphocytes were additionally stained for B cells (CD19) and T cells (CD4, CD8). Activated monocytes were detected by staining for MHC class II intensity (BD BioSciences). CHOP, IL-2 and rituximab treatments were as described above.

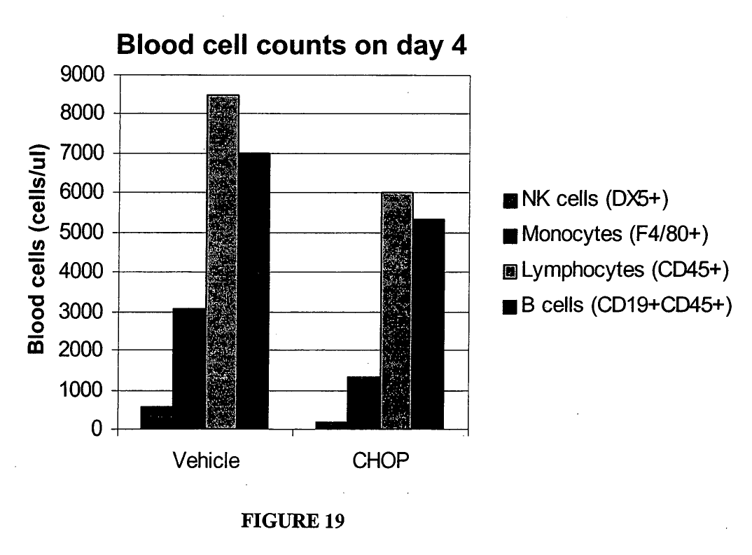

FIG. 19 shows the results of CHOP alone. Blood cell counts were conducted on day 4. As shown in FIG. 19, CHOP therapy depleted monocyte and lymphocyte populations in blood.

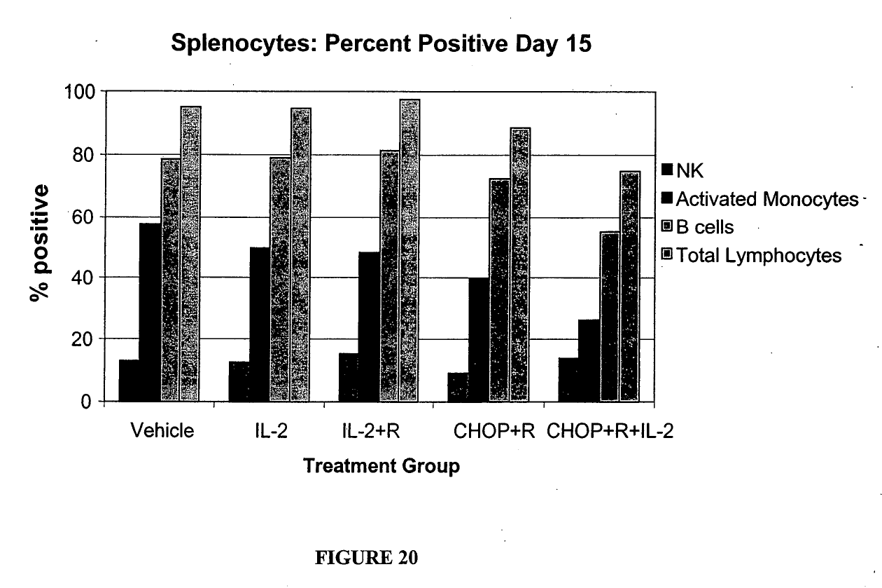

FIG. 20 shows the results of IL-2, IL-2+rituximab, CHOP+rituximab and CHOP+rituximab+IL-2. Cells were measured on day 15. As shown in the figure, enhanced splenic depletion of activated monocytes occurred following treatment with CHOP+rituximab+IL-2.

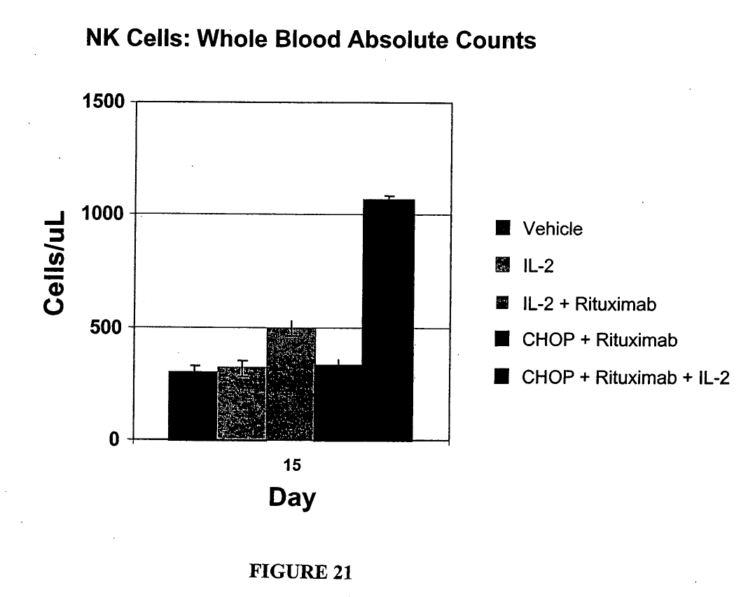

FIG. 21 shows the results of IL-2, IL-2+rituximab, CHOP+rituximab and CHOP+rituximab+IL-2. NK cells from whole blood were counted on day 15. As shown in the figure, there were increased blood NK cell numbers following treatment with CHOP+rituximab+IL-2.

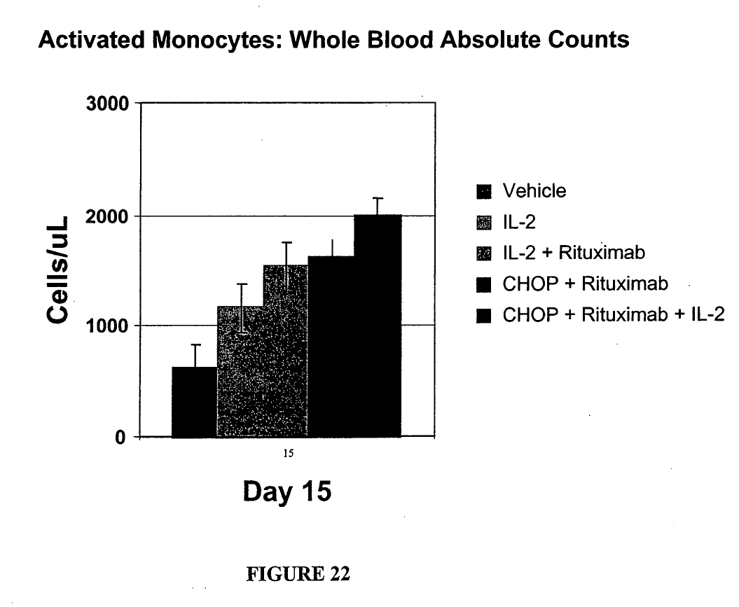

FIG. 22 shows the results of IL-2, IL-2+rituximab, CHOP+rituximab and CHOP+rituximab+IL-2. Activated monocytes from whole blood were counted on day 15. As shown in the figure, there were increased activated monocyte cell numbers following treatment with CHOP+rituximab+IL-2.

### Example 4

### Histological and Immunohistochemical Analysis of Daudi Tumors Following Therapy

The pharmacodynamics of drug treatments were also examined by measuring cellular trafficking of immune effector cells into Daudi tumors in vivo. Histological and immunochemical analysis of Daudi tumors from animals treated with various combinations of CHOP, IL-2 and rituximab was conducted. In particular, athymic nude BALB/c mice (10 mice/group) were acclimated and irradiated and mice were implanted with Daudi cells as described above. Treatment started when tumors were approximately 300 mm³. This was designated study day 1. CHOP, IL-2 and rituximab treatments were administered as follows: CHOP, day 1; rituximab, days 1 and 8; IL-2, days 8, 10 and 12. Tumors were excised and fixed in 10% neutral buffered formalin, transferred to 70% ethanol, and subsequently processed for paraffin embedding using an Excelsior tissue processor (Thermo Electron Corporation, Pittsburgh, Pa.). Tissue sections (4 μm) were cut on a rotary microtome (RM2235, Leica Microsystems, Nussloch, Germany). Hemotoxylin and eosin (H&E) stained sections were prepared. Immunohistochemical stainings were performed using a Discovery XT automated slide staining system (Ventana Medical Systems, Tucson, Ariz.). The primary antibodies used were: F4/80 (Serotec) for detection of monocytes and macrophages, anti-perforin antibody (1:800 dilution, Research Diagnostics, Inc., Flanders, N.J.) for NK cells, cleaved caspase 3 (Ab-2, 1:10 dilution, Oncogene Research Products, Boston, Mass.) for apoptosis, Ki-67 (K-2, neat, Ventana Medical Systems) for cellular proliferation rate, and a rabbit IgG1 (ChromPure, Jackson ImmunoResearch Laboratories, Inc., West Grove, Pa.) for the isotype control. Heat-induced epitope retrieval was performed using CC1 (Ventana Medical Systems). Samples were then incubated with the appropriate secondary antibody (rabbit anti-mouse IgG1 biotinylated antibody, 1:100 dilution, Research Diagnostics; or goat anti-rabbit IgG biotinylated antibody, 1:100 dilution, Jackson ImmunoResearch Laboratories). A horseradish peroxidase-labelled streptavidin biotin system with 3-3′-diaminobenzidine chromogen (Ventana Medical Systems) was used for localization. Sections were counterstained with hematoxylin to enhance visualization of tissue morphology.

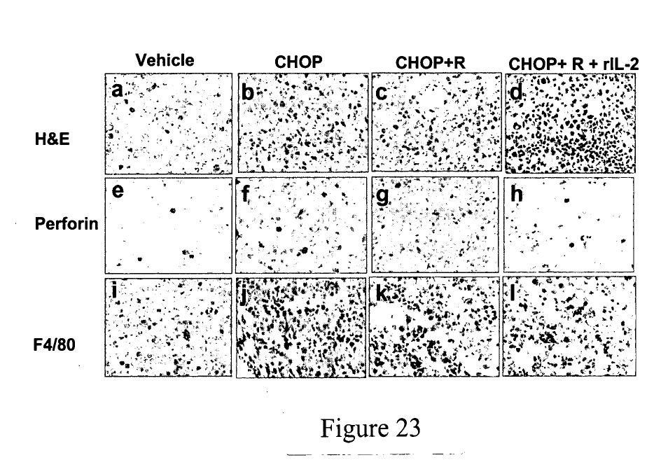

H&E analyses showed densely packed tumor cells, with few degenerating cells in the vehicle-treated group, and slightly higher numbers in the CHOP and CHOP-R groups (FIGS. 23a-c). CHOP-R/IL-2 tumors displayed degenerating tissue, infiltrating mononuclear cells, and fibrotic interstitial tissue in >50% of the tumor area (FIG. 23d). Activated immune effector cells, presumed NK/LAK cells, were identified by perforin staining (T cells are absent in BALB/c nude mice). Very few NK cells sparsely distributed in vehicle, CHOP, or CHOP-R treated tumors (FIGS. 23e-g), with marginally higher numbers of NK cells were detected in CHOP-R/IL-2 treatment groups (FIG. 23h). Macrophages were detected in tumors using F4/80 immunostaining. In striking contrast to the sparse NK cell infiltration, increased numbers of macrophages were found to be deposited in tumors treated with rituximab, IL-2+R, CHOP-R, and CHOP-R+IL-2 and in most cases appeared predominantly at the tumor periphery and/or contiguous to areas of degenerating cells (FIGS. 23i-l).

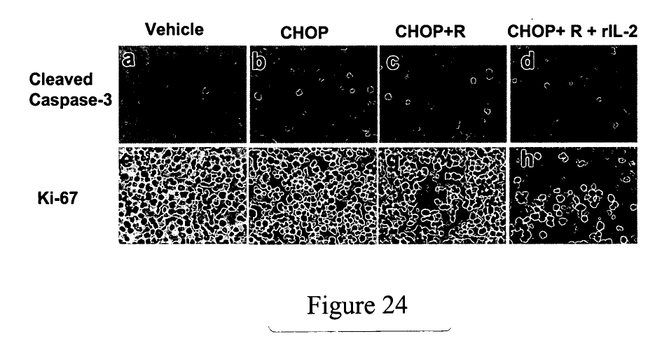

To address the mechanism of Daudi tumor responses to CHOP-R/IL-2 therapy, tumor sections were stained for markers of apoptotic and proliferating cells (FIGS. 24a-h). Tumor-cell death was assessed by cleaved caspase-3 immunostaining as a marker of apoptosis (FIGS. 24a-d). Although the numbers of cleaved caspase-3 positive cells were marginally increased in all drug-treated groups, given the higher areas of necrosis with IL-2/CHOP-R therapy, the numbers of apoptotic cells detected with IL-2/CHOP-R treatment were appreciably higher than other treatments per area of viable tissue. In addition, Ki67 staining of sections as a marker of tumor cell proliferation confirmed an enhanced antitumor effect with IL-2/CHOP-R therapy, concomitant with the potent and rapid early responses by day 15 (FIGS. 24e-h). Decreased levels of Ki67 were also evidenced in the IL-2/R (not shown) and CHOP-R treatment groups, but were not as pronounced as the IL-2/CHOP-R treatment group (FIG. 24h).

In summary, administration of CHOP+rituximab+IL-2 as detailed above induced apoptosis and decreased proliferative activity in Daudi tumors. The mechanism of CHOP+rituximab+IL-2 was found to correlate well with histological and IHC endpoints indicating increased antiproliferative and apoptotic activity against Daudi tumors.

### Example 5

### Assessment of Synergistic Activity in Groups Treated with Combination Therapy

Several studies of these agents and combinations of these agents were performed in the Daudi lymphoma tumor model. The data from these multiple experiments were then pooled together for further analysis (Table 10). In vivo drug synergy was evaluated from the response index of combination treatments using multiple drug effect analyses.

TABLE 10
Efficacy of IL-2, rituximab, chemotherapy, or their combination in the
Daudi xenograft model.

| | | | Tumor | |
|---|---|---|---|---|
| | | Growth | response | |
| | | delay | RR; CR/PR | Drug |
| Treatment^{a} | TGI | (days) | (n) | Interaction^{b} |
| IL-2 | 14%, p > 0.05 | 2 | 5%; 2/1 (60) | |
| Rituximab | 63%, p < 0.001 | 22 | 15%; 3/3 (40) | |
| Cyclophosphamide | 2%, p > 0.05 | 1 | 0%; 0/0 (10) | |
| Doxorubicin | 9%, p > 0.05 | 2 | 0%; 0/0 (10) | |
| Vincristine | 50%, p = 0.002 | 12 | 0%; 0/0 (10) | |
| Prednisone | 10%, p > 0.05 | 2 | 10%; 1/0 (10) | |
| CHOP | 78%, p < 0.001 | 17 | 17%; 4/1 (30) | Synergistic |
| IL-2 + Cyclophosphamide | 39%, p = 0.018 | 7 | 0%; 0/0 (10) | Additive |
| IL-2 + Doxorubicin | 26%, p = 0.082 | 5 | 0%; 0/0 (10) | Additive |
| IL-2 + Vincristine | 66%, p < 0.001 | 15 | 10%; 1/0 (10) | Additive |
| IL-2 + Prednisone | 32%, p = 0.044 | 5 | 10%; 1/0 (10) | Additive |
| IL-2 + CHOP | 93%, p < 0.001 | 26 | 40%; 7/1 (20) | Synergistic |
| IL-2 + Rituximab | 94%, p < 0.001 | >100 | 38%; 14/1 (40) | Synergistic |
| CHOP + Rituximab | 99%, p < 0.001 | 87 | 40%; 8/0 (20) | Synergistic |
| CHOP + Rituximab + IL-2 | >99%, p < 0.001 | >146 | 95%; 19/0 (20) | Synergistic |

^{a}n = 10 mice/group in each study. In cases where treatments were tested in multiple independent studies, data was combined. In these studies, treatments were initiated when tumors were between 100 and 250 mm³.
^{b}Synergistic effects were defined when the ratio of expected % tumor growth inhibition of combination therapy (% T/Cexp = % T/C treatment 1 × % T/C treatment 2) divided by observed % T/C (% T/Cobs) of the combination treatment was >1. Additive effects were defined when % T/Cexp/% T/Cobs = 1, and antagonism when % T/Cexp/% T/Cobs <1.

In the BALB/c nude Daudi model, single agent cyclophosphamide, doxorubicin, and prednisone produced marginal tumor growth inhibition (<15%) and were not statistically different from vehicle treatment (Table 10). Vincristine was effective in this Daudi model, eliciting a 50% tumor growth inhibition (P=0.002). When chemotherapeutic agents were administered as the CHOP combination, statistically significant tumor growth inhibition (78%, P<0.001) and tumor responses (4PR, 1CR; 17% response rate) were observed (Table 10). IL-2 monotherapy was well-tolerated and produced 14% tumor growth inhibition when administered thrice weekly at 1 mg/kg s.c. (P>0.05). Combinations of IL-2 with either cyclophosphamide, doxorubicin, vincristine, or prednisone were evaluated. All these combination regimens resulted in tumor growth inhibitory effects that were additive when compared with each agent alone. No indication of synergy was observed. IL-2 combined with the CHOP regimen demonstrated incremental tumor growth inhibition when compared with single agents, indicative of drug potentiation (combination therapy % T/C_{expected}/% T/C_{observed}˜3) (Table 10).

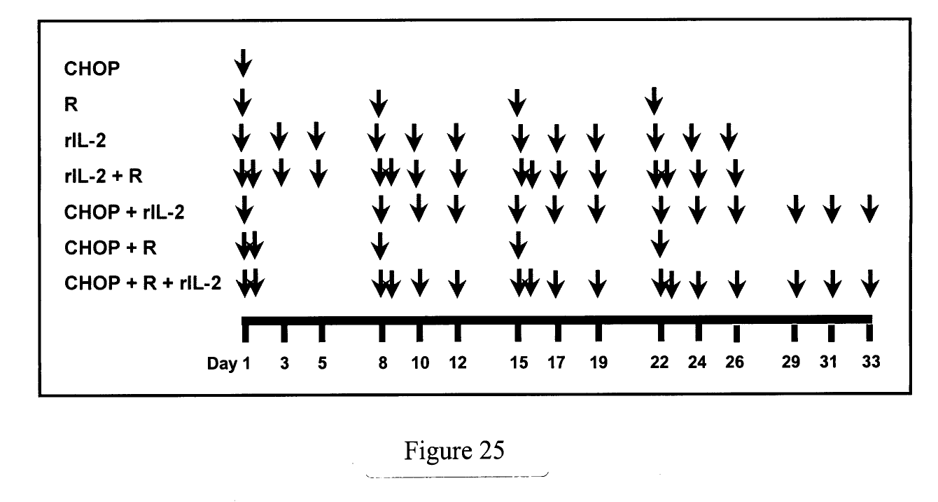

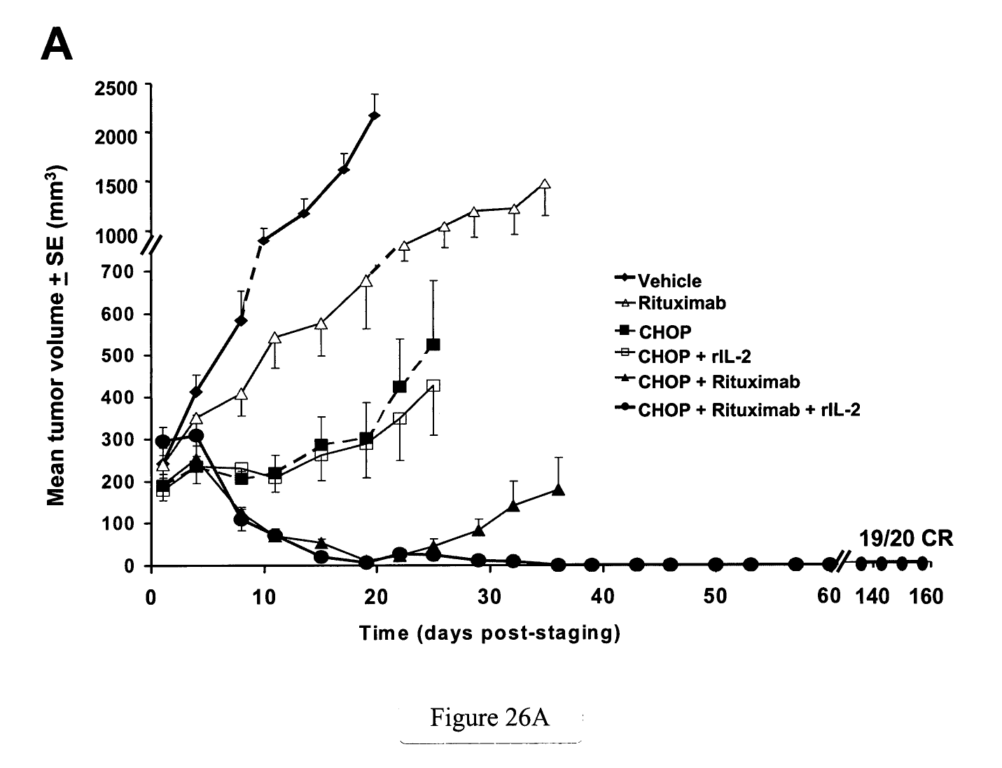

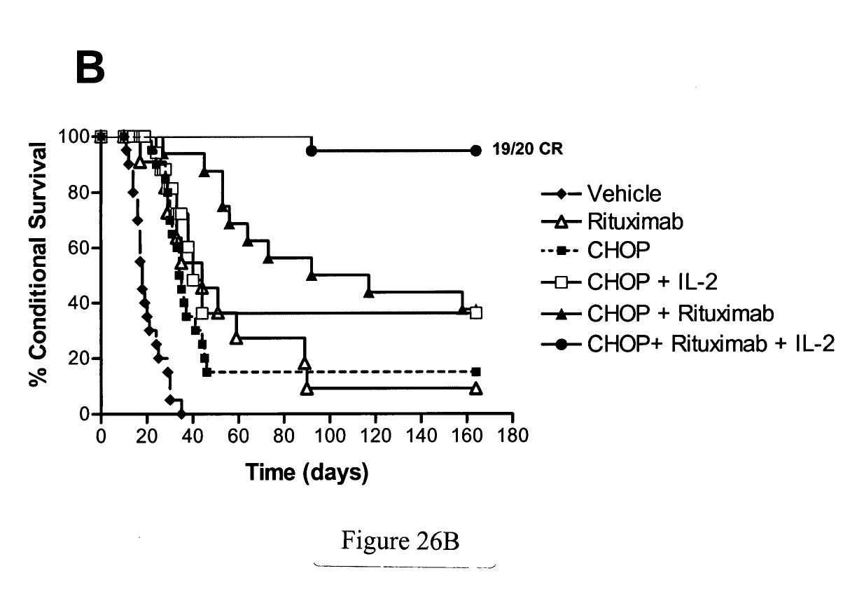

The combinations of IL-2 with rituximab treatment demonstrated synergistic activity when compared to the single agents. Addition of IL-2 to CHOP plus Rituxan combination treatment was evaluated to determine if IL-2 enhances the antitumor activity of CHOP-R (FIG. 25). Strikingly, the addition of IL-2 to CHOP-R therapy was curative in 19/20 mice; evidence of complete responses was observed by day 36 and was durable up to at least day 160 post-initiation of drug treatment (Table 10, FIG. 26A). Complete responses were confirmed using immunohistochemical staining with anti-human mitochondrial antibody for residual human Daudi cells at the site of tumor implantation on day 160 when the experiments were terminated. Kaplan-Meir analyses of survival as defined by time to 1000 mm³ tumor volume was statistically significant compared to all single agents or dual treatment groups (FIG. 26B). All treatment regimens were well tolerated, with no drug-related adverse events or significant body weight loss.

The data in these examples show that IL-2 enhances overall tumor efficacy and durability of responses when administered in combination with CHOP-R, or in combination with rituximab as maintenance therapy following CHOP-R treatment in a Daudi xenograft model of human low-grade CD20⁺ B-cell lymphoma in BALB/c nude mice. The most striking finding was that IL-2/CHOP-R therapy was curative in 95% of treated mice, with significant improvement in efficacy compared to all iterations of dual combinations, particularly the current standard agents, CHOP and rituximab. These data were also supported by analyses of drug additivity, indicating that addition of IL-2 synergistically benefits CHOP-R therapy.

What is claimed is:

1. A method for treating a B-cell lymphoma comprising administering to a subject in need thereof a therapeutically effective amount of (a) a chemotherapeutic agent; (b) an IL-2; and, optionally, (c) an anti-CD20 antibody or antigen-binding fragment thereof.

2. The method of claim 1, wherein the chemotherapeutic agent comprises one or more constituents selected from the group consisting of (a) cyclophosphamide, (b) doxorubicin, (c) vincristine, (d) prednisone and (e) combinations of cyclophosphamide, doxorubicin, vincristine and prednisone.

3. The method of claim 2, wherein said chemotherapeutic agent comprises cyclophosphamide.

4. The method of claim 2, wherein said chemotherapeutic agent comprises doxorubicin.

5. The method of claim 2, wherein said chemotherapeutic agent comprises vincristine.

6. The method of claim 2, wherein said chemotherapeutic agent comprises prednisone.

7. The method of claim 2, wherein said chemotherapeutic agent comprises cyclophosphamide, doxorubicin, vincristine and prednisone (CHOP).

8. The method of claim 1, wherein said antibody is an immunoglobulin G1 (IgG1) monoclonal antibody.

9. The method of claim 8, wherein said antibody is rituximab.

10. The method of claim 1, wherein said IL-2 is recombinantly produced IL-2 comprising an amino acid sequence with at least 70% sequence identity to the amino acid sequence for human IL-2.

11. The method of claim 10, wherein said IL-2 comprises an amino acid sequence with at least 80% sequence identity to the amino acid sequence for human IL-2.

12. The method of claim 10, wherein said IL-2 comprises an amino acid sequence with at least 90% sequence identity to the amino acid sequence for human IL-2.

13. The method of claim 10, wherein said IL-2 comprises an amino acid sequence with at least 95% sequence identity to the amino acid sequence for human IL-2.

14. The method of claim 10, wherein said IL-2 is des-alanyl-1, serine-125 human interleukin-2 (aldesleukin).

15. The method of claim 1, wherein said B cell lymphoma is low grade non-Hodgkin's lymphoma (NHL).

16. The method of claim 1, wherein said B cell lymphoma is intermediate grade non-Hodgkin's lymphoma (NHL).

17. The method of claim 1, wherein said B cell lymphoma is high grade non-Hodgkin's lymphoma (NHL).

18. The method of claim 1, wherein multiple therapeutically effective doses of the IL-2 and the anti-CD20 antibody are administered to said subject.

19. The method of claim 18, wherein said IL-2 is administered according to a twice-a-week or three-times-a-week dosing regimen.

20. The method of claim 18, wherein said IL-2 is administered subcutaneously.

21. The method of claim 18, wherein said anti-CD20 antibody is administered according to a once-a-week dosing regimen.

22. The method of claim 1, wherein said anti-CD20 antibody is administered intravenously.

23. The method of claim 1, wherein said chemotherapeutic agent is administered intravenously.

24. The method of claim 1, wherein said chemotherapeutic agent is administered orally.

25. The method of claim 1, wherein multiple therapeutically effective doses of the chemotherapeutic agent and the anti-CD20 antibody are administered to said subject.

26. The method of claim 25, wherein the chemotherapeutic agent is CHOP.

27. The method of claim 25, wherein multiple therapeutically effective doses of the IL-2 and the anti-CD20 antibody are administered after administration of the chemotherapeutic agent and the anti-CD20 antibody.

28. The method of claim 27, wherein the chemotherapeutic agent is CHOP.

29. The method of claim 1, wherein multiple therapeutically effective doses of said IL-2 are administered after administration of said chemotherapeutic agent.

30. The method of claim 29, wherein multiple therapeutically effective doses of said IL-2 are administered to said subject for a time period sufficient to effect immune reconstitution.

31. The method of claim 29, wherein the chemotherapeutic agent is CHOP.

32. The method of claim 1, wherein multiple therapeutically effective doses of the chemotherapeutic agent and the IL-2 are administered to said subject.

33. The method of claim 32, wherein the chemotherapeutic agent is CHOP.

34. The method of claim 1, wherein multiple therapeutically effective doses of the chemotherapeutic agent, the anti-CD20 antibody, and the IL-2 are administered to said subject.

35. The method of claim 34, wherein the chemotherapeutic agent is CHOP.

36. A method for treating low grade/follicular non-Hodgkin's lymphoma (NHL) comprising administering to a subject in need thereof a therapeutically effective amount of (a) CHOP; (b) des-alanyl-1, serine-125 human interleukin-2 (aldesleukin); and, optionally, (c) rituximab.

37. The method of claim 36, wherein multiple therapeutically effective doses of aldesleukin and rituximab are administered to said subject.

38. The method of claim 36, wherein multiple therapeutically effective doses of aldesleukin and CHOP are administered to said subject.

39. The method of claim 36, wherein multiple therapeutically effective doses of aldesleukin, CHOP, and rituximab are administered to said subject.

40. The method of claim 36, wherein said aldesleukin is administered according to a twice-a-week or three-times-a-week dosing regimen.

41. The method of claim 40, wherein said aldesleukin is administered subcutaneously.

42. The method of claim 36, wherein said rituximab is administered according to a once-a-week dosing regimen.

43. The method of claim 36, wherein said CHOP is administered intravenously.

44. The method of claim 36, wherein multiple therapeutically effective doses of said aldesleukin are administered after administration of said CHOP.

45. The method of claim 36, wherein multiple therapeutically effective doses of the CHOP and the rituximab are administered to said subject.

46. The method of claim 45, wherein multiple therapeutically effective doses of the aldesleukin and the rituximab are administered after administration of the CHOP and the rituximab.
